(19) United States
(12) Patent Application Publication
(10) Pub. No.: US 2023/0167508 A1
(43) Pub. Date: Jun. 1, 2023

(54) CELL-FREE DNA SIZE DETECTION

(71) Applicant: AccuraGen Holdings Limited, Georgetown (KY)

(72) Inventors: Li WENG, Fremont, CA (US); Malek FAHAM, Burlingame, CA (US); Tobias WITTKOP, San Mateo, CA (US); Johnny WU, Castro Valley, CA (US)

(21) Appl. No.: 18/054,676

(22) Filed: Nov. 11, 2022

Related U.S. Application Data
(63) Continuation of application No. PCT/US2021/031815, filed on May 11, 2021.
(60) Provisional application No. 63/024,489, filed on May 13, 2020.
(60) Provisional application No. 63/054,617, filed on Jul. 21, 2020.
(60) Provisional application No. 63/065,375, filed on Aug. 13, 2020.
(60) Provisional application No. 63/106,741, filed on Oct. 28, 2020.

(51) Int. Cl. C12Q 1/6886

(52) U.S. Cl. CPC C12Q 1/6886; C12Q 1/6869

(57) ABSTRACT

Provided herein are methods of cell-free nucleic acid size analysis. For example, methods herein can comprise preparing a first single stranded DNA library from a plurality of cell-free nucleic acid molecules from a subject and preparing a second single stranded DNA library from a plurality of cell-free nucleic acid molecules from a control.

### CROSS-REFERENCE

This application is a continuation of PCT International Application No. PCT/US2021/031815 filed on May 11, 2021, which claims the benefit of U.S. Provisional Application No. 63/024,489, filed May 13, 2020, U.S. Provisional Application No. 63/054,617, filed Jul. 21, 2020, U.S. Provisional Application No. 63/065,375, filed Aug. 13, 2020, and U.S. Provisional Application No. 63/106,741, filed Oct. 28, 2020, each of which is incorporated herein by reference in its entirety.

### BACKGROUND

Detection and analysis of cell-free nucleic acids has emerged as important method for studying health of an individual. For example, increased levels of cell-free nucleic acids are observed in individuals that have cancer. In addition, analysis of cell-free nucleic acids in a pregnant person allow for non-invasive prenatal screening for chromosomal abnormalities in a fetus.

### SUMMARY

Provided herein are methods of cell-free DNA size detection and analysis. Disclosed herein is a method wherein a more significant size difference by using single strand library prep than double stranded library prep. For example, a difference in fragment size between late stage cancer and healthy may be 29 bp using a single strand DNA library prep, and 12 bp using double stranded prep.

Further disclosed herein is a method wherein a more significant is observed size difference between differentially methylated molecules between cancer and healthy cell-free DNA.

In one aspect, provided herein are methods for nucleic acid analysis, comprising: (a) preparing a first single-stranded deoxynucleic acid (DNA) library from a first plurality of nucleic acid molecules, the first plurality of nucleic acid molecules comprising single-stranded DNA and double-stranded DNA derived from a subject; (b) preparing a second single-stranded DNA library from a second plurality of nucleic acid molecules, the second plurality of nucleic acid molecules comprising single-stranded DNA and double-stranded DNA derived from a control; (c) using the first single-stranded DNA library to measure a first size distribution for at least a subset of the first plurality of nucleic acid molecules; (d) using the second single-stranded DNA library to measure a second size distribution for at least a subset of the second plurality of nucleic acid molecules; (e) using the first size distribution and the second size distribution to determine a difference in size between the first size distribution and the second size distribution. In some cases, (a) comprises denaturing the first plurality of nucleic acid molecules. In some cases, (b) comprises denaturing the second plurality of nucleic acid molecules. In some cases, (a) comprises ligating an adapter to 5′ ends, 3′ ends or 5′ and 3′ ends of individual single stranded DNA molecules of the first plurality of nucleic acid molecules using a DNA ligase specific for single stranded DNA. In some cases, (b) comprises ligating an adapter to 5′ ends, 3′ ends or 5′ and 3′ ends of individual single stranded DNA molecules of the second plurality of nucleic acid molecules using a DNA ligase specific for single stranded DNA. In some cases, (a) comprises (i) circularizing individual single stranded DNA molecules of the first plurality of nucleic acid molecules to form a first plurality of circular nucleic acid molecules; and (ii) amplifying the first plurality of circular nucleic acid molecules to yield a first plurality of amplified nucleic acid molecules. In some cases, (b) comprises (i) circularizing individual single stranded DNA molecules of the second plurality of nucleic acid molecules to form a second plurality of circular nucleic acid molecules; and (ii) amplifying the second plurality of circular nucleic acid molecules to yield a second plurality of amplified nucleic acid molecules.

The method of claim 6 or claim 7, wherein circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules. In some cases, the amplification is performed by a polymerase having strand displacement activity. In some cases, the amplification is performed by a polymerase that does not have strand displacement activity. In some cases, the amplification comprises contacting the individual nucleic acid molecules to an amplification reaction mixture comprising random primers. In some cases, the amplification comprises contacting the individual nucleic acid molecules to an amplification reaction mixture comprising target-specific primers. In some cases, the method further comprises enriching the first plurality of nucleic acid molecules or the second plurality of nucleic acid molecules for one or more target sequences. In some cases, the enriching is performed with one or more primers or capture probes. In some cases, the enriching is performed with one or more antibodies or fragments thereof. In some cases, the size distribution for the first single-stranded DNA library or the second single-stranded DNA library comprises sequencing the first single-stranded DNA library or the second single-stranded DNA library. In some cases, sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing. In some cases, the method further comprises identifying an individual nucleic acid molecule of the single-stranded DNA library or the second single stranded library as having a genomic feature. In some cases, the genomic feature comprises an epigenetic modification. In some cases, the epigenetic modification is selected from the group consisting of methylation, phosphorylation, ubiquitination, sumoylation, acetylation, ribosylation, citrullination, and fragmentation. In some cases, the genomic feature comprises a copy number variation (CNV), a single nucleotide variant (SNV), an insertion, a deletion, a translocation, or a combination thereof. In some cases, the first plurality of nucleic acid molecules or the second plurality of nucleic acid molecules is derived from a cell-free biological sample. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, the first plurality of nucleic acids comprises tumor nucleic acids. In some cases, the second plurality of nucleic acids is derived from a healthy control. In some cases, the subject is determined to be at risk of or to have a disease when the difference is greater than a predetermined threshold. In some cases, the subject is determined to be at risk of or to have a disease when an average of the first size distribution is less than an average of the second size distribution. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some cases, the method further comprises administering a therapeutic to the subject. In some cases, the method further comprises recommending additional cancer monitoring to the subject. In some cases, the method further comprises using the difference to monitor the subject for a progression or a regression of the disease.

In another aspect, there are provided methods for nucleic acid analysis, comprising: (a) preparing a single-stranded deoxynucleic acid (DNA) library from a plurality of nucleic acid molecules the plurality comprising single-stranded DNA and double-stranded DNA derived from a subject; (b) identifying an individual nucleic acid molecule of the single-stranded DNA library as having a genomic feature; (c) measuring a size for the individual nucleic acid molecule; (d) associating the genomic feature with a disease based on the size of the individual nucleic acid molecule. In some cases, (a) comprises denaturing the plurality of nucleic acid molecules. In some cases, (a) comprises ligating an adapter to 5′ ends, 3′ ends or 5′ and 3′ ends of individual single stranded DNA molecules of the plurality of nucleic acid molecules using a DNA ligase specific for single stranded DNA. In some cases, (a) comprises (i) circularizing individual single stranded DNA molecules of the plurality of nucleic acid molecules to form a plurality of circular nucleic acid molecules; and (ii) amplifying the plurality of circular nucleic acid molecules to yield a plurality of amplified nucleic acid molecules. In some cases, circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules. In some cases, the amplification is performed by a polymerase having strand displacement activity. In some cases, the amplification is performed by a polymerase that does not have strand displacement activity. In some cases, the amplification comprises contacting the individual nucleic acid molecules to an amplification reaction mixture comprising random primers. In some cases, the amplification comprises contacting the individual nucleic acid molecules to an amplification reaction mixture comprising target-specific primers. In some cases, the method further comprises enriching the plurality of nucleic acid molecules for one or more target sequences. In some cases, the enriching is performed with one or more primers or capture probes. In some cases, the enriching is performed with one or more antibodies or fragments thereof. In some cases, (b) comprises sequencing the single-stranded DNA library. In some cases, (c) comprises sequencing the single-stranded DNA library. In some cases, sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing. In some cases, the genomic feature comprises an epigenetic modification. In some cases, the epigenetic modification is selected from the group consisting of methylation, phosphorylation, ubiquitination, sumoylation, acetylation, ribosylation, citrullination, and fragmentation. In some cases, the genomic feature comprises a copy number variation (CNV), a single nucleotide variant (SNV), an insertion, a deletion, a translocation, or a combination thereof. In some cases, the plurality of nucleic acid molecules is derived from a cell-free biological sample. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, the plurality of nucleic acids comprises tumor nucleic acids. In some cases, the individual nucleic acid molecule having the genomic feature is determined to be associated with a disease when the individual nucleic acid molecule has an average size below a predetermined threshold. In some cases, the individual nucleic acid molecule having the genomic feature is determined to be associated with disease when the individual nucleic acid molecule having the genomic feature has an average size smaller than a nucleic acid molecule without the genomic feature. In some cases, the subject is determined to be at risk of or to have a disease when the individual nucleic acid molecule having the genomic feature has a size below a predetermined threshold. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some cases, the method further comprises administering a therapeutic to the subject. In some cases, the method further comprises recommending additional cancer monitoring to the subject. In some cases, the method further comprises using the genomic feature or the size to monitor the subject for a progression or a regression of the disease.

In a further aspect, there are provided methods for nucleic acid analysis, comprising: (a) preparing a single-stranded deoxynucleic acid (DNA) library from a plurality of nucleic acid molecules the plurality comprising single-stranded DNA and double-stranded DNA derived from a subject; (b) identifying an individual nucleic acid molecule of the single-stranded DNA library as having a genomic feature; (c) identifying at least a 5′ end or a 3′ end for the individual nucleic acid molecule; (d) associating the genomic feature with a disease based on the 5′ end or the 3′ end of the individual nucleic acid molecule. In some cases, (a) comprises denaturing the plurality of nucleic acid molecules. In some cases, (a) comprises ligating an adapter to 5′ ends, 3′ ends or 5′ and 3′ ends of individual single stranded DNA molecules of the plurality of nucleic acid molecules using a DNA ligase specific for single stranded DNA. In some cases, (a) comprises (i) circularizing individual single stranded DNA molecules of the plurality of nucleic acid molecules to form a plurality of circular nucleic acid molecules; and (ii) amplifying the plurality of circular nucleic acid molecules to yield a plurality of amplified nucleic acid molecules. In some cases, circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules. In some cases, the amplification is performed by a polymerase having strand displacement activity. In some cases, the amplification is performed by a polymerase that does not have strand displacement activity. In some cases, the amplification comprises contacting the individual nucleic acid molecules to an amplification reaction mixture comprising random primers. In some cases, the amplification comprises contacting the individual nucleic acid molecules to an amplification reaction mixture comprising target-specific primers. In some cases, the method further comprises enriching the plurality of nucleic acid molecules for one or more target sequences. In some cases, the enriching is performed with one or more primers or capture probes. In some cases, the enriching is performed with one or more antibodies or fragments thereof. In some cases, (b) comprises sequencing the single-stranded DNA library. In some cases, (c) comprises sequencing the single-stranded DNA library. In some cases, sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing. In some cases, the genomic feature comprises an epigenetic modification. In some cases, the epigenetic modification is selected from the group consisting of methylation, phosphorylation, ubiquitination, sumoylation, acetylation, ribosylation, citrullination, and fragmentation. In some cases, the genomic feature comprises a copy number variation (CNV), a single nucleotide variant (SNV), an insertion, a deletion, or a combination thereof. In some cases, the plurality of nucleic acid molecules is derived from a cell-free biological sample. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, the plurality of nucleic acids comprises tumor nucleic acids. In some cases, the subject is determined to be at risk of or to have a disease when the 5′ end or the 3′ end of the individual nucleic acid molecule having the genomic feature has a predetermined sequence. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some cases, the method further comprises administering a therapeutic to the subject. In some cases, the method further comprises recommending additional cancer monitoring to the subject. In some cases, the method further comprises using the genomic feature or the 5′ end or 3′ end to monitor the subject for a progression or a regression of the disease.

In another aspect, there are provided methods for preparing a library enriched for cancer-derived nucleic acids, the method comprising: subjecting a population of nucleic acid molecules from a cell-free biological sample to enrichment nucleic acid molecules having sizes less than a predetermined threshold, thereby creating a library enriched for cancer-derived nucleic acids. In some cases, the enrichment comprises size selection of the population of nucleic acid molecules. In some cases, size selection comprises a bead purification or a gel purification of the population of nucleic acid molecules. In some cases, the enrichment comprises (a) ligating adapters to the population of nucleic acid molecules and (b) contacting the population of nucleic acid molecules with a cleaving agent. In some cases, the cleaving agent comprises a nuclease. In some cases, the enrichment comprises circularizing individual nucleic acid molecules of the population of nucleic acid molecules using an enzyme that favors small fragments. In some cases, the enrichment comprises (a) circularizing individual nucleic acid molecules of the population of nucleic acid molecules; and (b) contacting the population of nucleic acid molecules with a cleaving agent. In some cases, circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules. In some cases, the method further comprises amplifying the nucleic acid molecules having sizes less than the predetermined threshold. In some cases, the amplifying is performed by a polymerase having strand displacement activity. In some cases, the amplifying is performed by a polymerase that does not have strand displacement activity. In some cases, the amplifying comprises contacting the individual nucleic acid molecules to an amplification reaction mixture comprising random primers. In some cases, the amplifying comprises contacting the individual nucleic acid molecules to an amplification reaction mixture comprising target-specific primers. In some cases, the method further comprises enriching the nucleic acid molecules having sizes less than the predetermined threshold for one or more target sequences. In some cases, the enriching is performed with one or more primers or capture probes. In some cases, the enriching is performed with one or more antibodies or fragments thereof. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof.

In one aspect, there are provided methods of cell-free nucleic acid size analysis, the method comprising: (a) preparing a first single stranded deoxynucleic acid (DNA) library from a plurality of cell-free nucleic acid molecules from a subject; (b) preparing a second single stranded DNA library from a plurality of cell-free nucleic acid molecules from a control; (c) using the first single stranded library to measure a first size for at least a subset of the plurality of nucleic acid molecules from the subject; (d) using the second single stranded library to measure a second size for at least a subset of the plurality of nucleic acid molecules from the control; (e) detecting a difference in the first size and the second size when the subject has or is at risk of having cancer. In some embodiments, the difference is enhanced compared to a method using a double stranded library. In some embodiments, a methylation status is determined for the first single stranded library and the second single stranded library. In some embodiments, a copy number variation (CNV) is determined for the first single stranded library and the second single stranded library. In some embodiments, the subject is determined to be at risk of or to have a cancer when the difference is greater than a predetermined threshold. In some embodiments, the subject is determined to be at risk of or to have a cancer when the first size is less than the second size.

In another aspect, there are provided methods for nucleic acid analysis, comprising: (a) preparing a single-stranded deoxynucleic acid (DNA) library from a plurality of nucleic acid molecules the plurality comprising single-stranded DNA and double-stranded DNA derived from a subject; (b) identifying at least a 5′ end or a 3′ end for the individual nucleic acid molecule; (c) associating the 5′ end or the 3′ end of the individual nucleic acid molecule with a disease. In some embodiments, (a) comprises denaturing the plurality of nucleic acid molecules. In some embodiments, (a) comprises ligating an adapter to 5′ ends, 3′ ends or 5′ and 3′ ends of individual single stranded DNA molecules of the plurality of nucleic acid molecules using a DNA ligase specific for single stranded DNA. In some embodiments, (a) comprises (i) circularizing individual single stranded DNA molecules of the plurality of nucleic acid molecules to form a plurality of circular nucleic acid molecules; and (ii) amplifying the plurality of circular nucleic acid molecules to yield a plurality of amplified nucleic acid molecules. In some embodiments, circularizing comprises ligating 5′ ends of the individual nucleic acid molecules to 3′ ends of the individual nucleic acid molecules. In some embodiments, circularizing comprises coupling adapters to 3′ ends, 5′ ends, or both 5′ ends and 3′ ends of the individual nucleic acid molecules. In some embodiments, the amplification is performed by a polymerase having strand displacement activity. In some embodiments, the amplification is performed by a polymerase that does not have strand displacement activity. In some embodiments, the amplification comprises contacting the individual nucleic acid molecules to an amplification reaction mixture comprising random primers. In some embodiments, the amplification comprises contacting the individual nucleic acid molecules to an amplification reaction mixture comprising target-specific primers. In some embodiments, the method further comprises enriching the plurality of nucleic acid molecules for one or more target sequences. In some embodiments, the enriching is performed with one or more primers or capture probes. In some embodiments, the enriching is performed with one or more antibodies or fragments thereof. In some embodiments, (b) comprises sequencing the single-stranded DNA library. In some embodiments, sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing. In some embodiments, the method further comprises identifying an individual nucleic acid molecule of the single-stranded DNA library as having a genomic feature. In some embodiments, the genomic feature comprises an epigenetic modification. In some embodiments, the epigenetic modification is selected from the group consisting of methylation, phosphorylation, ubiquitination, sumoylation, acetylation, ribosylation, citrullination, and fragmentation. In some embodiments, the genomic feature comprises a copy number variation (CNV), a single nucleotide variant (SNV), an insertion, a deletion, or a combination thereof. In some embodiments, the plurality of nucleic acid molecules is derived from a cell-free biological sample. In some embodiments, the cell-free biological sample comprises a bodily fluid. In some embodiments, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some embodiments, the plurality of nucleic acids comprises tumor nucleic acids. In some embodiments, the subject is determined to be at risk of or to have a disease when the 5′ end or the 3′ end of the individual nucleic acid molecule has a predetermined sequence. In some embodiments, the disease is cancer. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some embodiments, the method further comprises administering a therapeutic to the subject. In some embodiments, the method further comprises recommending additional cancer monitoring to the subject. In some embodiments, the method further comprises using the 5′ end or 3′ end to monitor the subject for a progression or a regression of the disease. In some embodiments, (a) does not comprise end repair.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows double stranded deoxyribonucleic acid (DNA) preparation size difference between cancer cell-free DNA and healthy cell-free DNA.

FIG. 2 shows single stranded DNA preparation size difference between cancer cell-free DNA and healthy cell-free DNA.

FIG. 3 shows copy number variation (CNV) signals stand out in small fragments captured by single stranded library preparation.

FIG. 4 shows single strand DNA preparation captures size difference between differentially methylated sites in cancer and healthy cell-free DNA.

FIG. 5 shows data illustrating the size difference between mutant cfDNA molecules and wild type cfDNA molecules in a method utilizing single strand DNA library preparation. This size difference is greater than a difference observed in methods utilizing double stranded DNA library preparation.

FIG. 6 shows data illustrating size difference between differentially methylated sites in cancer patient blood samples using single stranded library preparation.

FIG. 7 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “about” or “approximately” means within an acceptable error range for the particular value as determined by one of ordinary skill in the art, which may depend in part on how the value is measured or determined, i.e., the limitations of the measurement system. For example, “about” can mean within 1 or more than 1 standard deviation, per the practice in the art. As another example, “about” can mean a range of up to 20%, up to 10%, up to 5%, or up to 1% of a given value. With respect to biological systems or processes, the term “about” can mean within an order of magnitude, such as within 5-fold or within 2-fold of a value. Where particular values are described in the application and claims, unless otherwise stated, the term “about” means within an acceptable error range for the particular value.

As used herein, the terms “polynucleotide”, “nucleotide”, “nucleotide sequence”, “nucleic acid” and “oligonucleotide” generally refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: deoxyribonucleic acids (DNA), ribonucleic acids (RNA), cell-free nucleic acids, cell-free DNA (cfDNA), circulating tumor DNA (ctDNA), coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “subject” as used herein, generally refers to an individual, such as a vertebrate. A vertebrate may be a mammal (e.g., a human). Mammals include, but are not limited to, murines, simians, humans, farm animals, sport animals, and pets. Tissues, cells, and their progeny of a biological entity obtained in vivo or cultured in vitro are also encompassed. The subject may be a patient. The subject may be symptomatic with respect to a disease (e.g., cancer). As an alternative, the subject may be asymptomatic with respect to the disease.

The term “early stage cancer” refers to a cancer that has not yet metastasized in an individual (i.e., the cancer has not left its initial location to spread to other locations). In some cases, an early stage cancer is “non-metastatic cancer.” The exact staging depends upon the type of cancer, details for which are provided herein.

The terms “tumor burden” and “tumor load,” as used herein, generally refer to a size of a tumor or an amount of a disease (e.g., cancer) in a body of a subject.

The term “healthy control,” as used herein, generally refers to a point of reference for a healthy state of a subject(s). The healthy control may be from a subject(s) not having or suspected of having a disease (e.g., cancer), or from a subject(s) having or suspected of having the disease but from a location that may otherwise be used as a non-disease reference (e.g., white blood cells). The healthy control may be a genome or a portion of a genome. The healthy control may be ribonucleic acid molecule(s) and/or deoxyribonucleic acid molecule(s).

The term “sample,” as used herein, generally refers to a sample derived from or obtained from a subject, such as a mammal (e.g., a human). The sample may be a biological sample. Samples may include, but are not limited to, hair, finger nails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject. The biological sample may be a cell-free (or cell free) biological sample.

The term “cell-free,” as used herein, generally refers to a sample derived from or obtained from a subject that is free from cells. Cell-free biological samples may include, but are not limited to, blood, serum, plasma, nasal swab or nasopharyngeal wash, saliva, urine, gastric fluid, tears, stool, mucus, sweat, earwax, oil, glandular secretion, bile, lymph, cerebrospinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions.

### Methods of Nucleic Acid Analysis of Sizes

Provided herein are method for nucleic acid analysis, such as nucleic acid size analysis. A method for nucleic acid analysis may comprise preparing a first single-stranded deoxynucleic acid (DNA) library from a first plurality of nucleic acid molecules, the first plurality of nucleic acid molecules comprising single-stranded DNA and double-stranded DNA derived from a subject. In parallel or subsequently, the method may comprise preparing a second single-stranded DNA library from a second plurality of nucleic acid molecules, the second plurality of nucleic acid molecules comprising single-stranded DNA and double-stranded DNA derived from a control. Next, the method may comprise using the first single-stranded DNA library to measure a first size distribution for at least a subset of the first plurality of nucleic acid molecules. In parallel or subsequently, the method may comprise using the second single-stranded DNA library to measure a second size distribution for at least a subset of the second plurality of nucleic acid molecules. Next, the library may comprise using the first size distribution and the second size distribution to determine a difference in size between the first size distribution and the second size distribution.

In an aspect of methods of nucleic acid analysis herein, methods may comprise preparing a single-stranded DNA library. In some cases, preparing the first single-stranded DNA library comprises denaturing the first plurality of nucleic acid molecules. In some cases, preparing the second single-stranded DNA library comprises denaturing the second plurality of nucleic acid molecules. In some cases, preparing the first single-stranded DNA library comprises ligating an adapter to 5′ ends, 3′ ends or 5′ and 3′ ends of individual single stranded DNA molecules of the first plurality of nucleic acid molecules using a DNA ligase specific for single stranded DNA. In some cases, preparing the second single-stranded DNA library comprises ligating an adapter to 5′ ends, 3′ ends or 5′ and 3′ ends of individual single stranded DNA molecules of the second plurality of nucleic acid molecules using a DNA ligase specific for single stranded DNA.

In some methods of preparing a single-stranded DNA library, individual single-stranded DNA molecules are circularized. In some cases, the individual single-stranded DNA molecules have been denatured. In some cases, the individual single-stranded DNA molecules have not been denatured. In some cases, preparing the first single-stranded DNA library comprises (i) circularizing individual single-stranded DNA molecules of the first plurality of nucleic acid molecules to form a first plurality of circular nucleic acid molecules; and (ii) amplifying the first plurality of circular nucleic acid molecules to yield a first plurality of amplified nucleic acid molecules. In some cases, preparing the second single-stranded DNA library comprises (i) circularizing individual single stranded DNA molecules of the second plurality of nucleic acid molecules to form a second plurality of circular nucleic acid molecules; and (ii) amplifying the second plurality of circular nucleic acid molecules to yield a second plurality of amplified nucleic acid molecules. In some cases, circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules. In some cases, amplification is performed by a polymerase having strand displacement activity. In some cases, amplification is performed by a polymerase that does not have strand displacement activity. In some cases, amplification comprises contacting the individual nucleic acid molecules to an amplification reaction mixture comprising random primers. In some cases, amplification comprises contacting the individual nucleic acid molecules to an amplification reaction mixture comprising target-specific primers.

In an aspect of methods of nucleic acid analysis provided herein, the single-stranded DNA library may comprise a small amount of contaminating double-stranded DNA from the original input DNA. In some cases, the first single-stranded DNA library comprises at most 5% double stranded DNA from the original input DNA. In some cases, the second single-stranded DNA library comprises at most 5% double stranded DNA from the original input DNA. In some cases, the first or second single-stranded DNA library comprises at most 1% double stranded DNA from the original input DNA. In some cases, the first or second single-stranded DNA library comprises at most 0.5% double stranded DNA from the original input DNA.

In an aspect of methods of nucleic acid analysis provided herein, the method may further comprise an enrichment step to enrich for one or more target sequences. In some cases, the method further comprises enriching the first plurality of nucleic acid molecules or the second plurality of nucleic acid molecules for one or more target sequences. In some cases, enriching is performed with one or more primers or capture probes. In some cases, enriching is performed with one or more antibodies or fragments thereof.

In an aspect of methods of nucleic acid analysis provided herein, methods may comprise measuring a size distribution for a single-stranded DNA library. In some cases, measuring the size distribution for the first single-stranded DNA library or the second single-stranded DNA library comprises sequencing the first single-stranded DNA library or the second single-stranded DNA library. In some cases, measuring the size distribution for the first single-stranded DNA library or the second single-stranded DNA library may include but are not limited to sequencing, bioanalyzer fragment analysis, PCR, qPCR, high throughput gel electrophoresis, high throughput capillary electrophoresis, and any other suitable methods that provide sizes of DNA fragments.

In an aspect of nucleic acid analysis provided herein, methods may comprise sequencing a single stranded DNA library. In some cases, sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing.

In an aspect of nucleic acid analysis provided herein, methods may comprise determining an epigenetic modification for the first single-stranded DNA library or the second single-stranded DNA library. In some cases, the epigenetic modification is selected from the group consisting of methylation, phosphorylation, ubiquitination, sumoylation, acetylation, ribosylation, citrullination, and fragmentation.

In an aspect of nucleic acid analysis provided herein, methods may comprise determining a copy number variation (CNV), a single nucleotide variant (SNV), an insertion, a deletion, a translocation, or a combination thereof, for the first single-stranded DNA library or the second single-stranded DNA library.

In an aspect of nucleic acid analysis provided herein, a plurality of nucleic acid molecules may be derived from a sample, such as a cell-free biological sample. In some cases, the first plurality of nucleic acid molecules or the second plurality of nucleic acid molecules is derived from a cell-free biological sample. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, a plurality of nucleic acids are derived from a subject suspected of having a disease, such as cancer. In some cases, a plurality of nucleic acids are derived from a healthy control. In some cases, the first plurality of nucleic acids comprises tumor nucleic acids. In some cases, the second plurality of nucleic acids is derived from a healthy control.

In an aspect of nucleic acid analysis provided herein, the analysis further provides risk of a disease or the presence of a disease in a subject. In some cases, the subject is determined to be at risk of or to have a disease when the difference in size is greater than a predetermined threshold. In some cases, the subject is determined to be at risk of or to have a disease when an average of the first size distribution is less than an average of the second size distribution. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some cases, the method further comprises administering a therapeutic to the subject. In some cases, the method further comprises recommending additional cancer monitoring to the subject. In some cases, the method further comprises using the difference to monitor the subject for a progression or a regression of the disease.

Further provided herein are methods of cell-free nucleic acid size analysis. In some cases, methods comprise preparing a first single stranded DNA library from a plurality of cell-free nucleic acid molecules from a subject and preparing a second single stranded DNA library from a plurality of cell-free nucleic acid molecules from a control. Next, in some cases, the first single stranded library is used to measure a first size for at least a subset of the plurality of nucleic acid molecules from the subject and the second single stranded library is used to measure a second size for at least a subset of the plurality of nucleic acid molecules from the control. Then, in some cases, a difference is detected in the first size and the second size when the subject has or is at risk of having cancer. In some cases, the difference is enhanced compared to a method using a double stranded library. In some cases, a methylation status is determined for the first single stranded library and the second single stranded library. In some cases, a copy number variation (CNV) is determined for the first single stranded library and the second single stranded library. In some cases, the subject is determined to be at risk of or to have a cancer when the difference is greater than a predetermined threshold. In some cases, the subject is determined to be at risk of or to have a cancer when the first size is less than the second size.

### Methods for Nucleic Acid Analysis of Genomic Features and Nucleic Acid End Analysis

In a further aspect, there are provided methods for nucleic acid analysis, comprising: preparing a single-stranded deoxynucleic acid (DNA) library from a plurality of nucleic acid molecules, the plurality of nucleic acid molecules comprising single-stranded DNA and double-stranded DNA derived from a subject. The method may then comprise identifying an individual nucleic acid molecule of the single-stranded DNA library as having a genomic feature. Alternatively or in combination, the method may then comprise identifying a 5′ end and/or a 3′ end of an individual nucleic acid molecule of the single-stranded DNA library. Next, the method may comprise measuring a size for the individual nucleic acid molecule. Then the method may comprise associating the genomic feature with a disease based on the size of the individual nucleic acid molecule. Alternatively or in combination, the method may comprise associating the 5′ end and/or the 3′ end of the individual nucleic acid molecule with a disease.

In an additional aspect, there are provided method for nucleic acid analysis, comprising preparing a single-stranded deoxynucleic acid (DNA) library from a plurality of nucleic acid molecules the plurality of nucleic acid molecules comprising single-stranded DNA and double-stranded DNA derived from a subject. Next, an individual nucleic acid molecule of the single-stranded DNA library may be identified as having a genomic feature. Then, at least a 5′ end or a 3′ end for the individual nucleic acid molecule may be identified. Then, the genomic feature may be associated with a disease based on the 5′ end or the 3′ end of the individual nucleic acid molecule.

In an aspect of methods of nucleic acid analysis herein, methods may comprise preparing a single-stranded DNA library. In some cases, preparing the single-stranded DNA library comprises denaturing the \ plurality of nucleic acid molecules. In some cases, preparing the single-stranded DNA library comprises ligating an adapter to 5′ ends, 3′ ends or 5′ and 3′ ends of individual single stranded DNA molecules of the plurality of nucleic acid molecules using a DNA ligase specific for single stranded DNA.

In some methods of preparing a single-stranded DNA library, individual single-stranded DNA molecules are circularized. In some cases, the nucleic acid sample is subjected to denaturation to create single-stranded DNA. In some cases, the nucleic acid sample is not subjected to denaturation. In some cases, preparing the single-stranded DNA library comprises (i) circularizing individual single-stranded DNA molecules of the first plurality of nucleic acid molecules to form a plurality of circular nucleic acid molecules; and (ii) amplifying the plurality of circular nucleic acid molecules to yield a first plurality of amplified nucleic acid molecules. In some cases, circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules. In some cases, amplification is performed by a polymerase having strand displacement activity. In some cases, amplification is performed by a polymerase that does not have strand displacement activity. In some cases, amplification comprises contacting the individual nucleic acid molecules to an amplification reaction mixture comprising random primers. In some cases, amplification comprises contacting the individual nucleic acid molecules to an amplification reaction mixture comprising target-specific primers.

In an aspect of methods of nucleic acid analysis provided herein, the single-stranded DNA library may comprise a small amount of contaminating double-stranded DNA from the original input DNA. In some cases, the single-stranded DNA library comprises at most 5% double stranded DNA from the original input DNA. In some cases, the single-stranded DNA library comprises at most 1% double stranded DNA from the original input DNA. In some cases, the single-stranded DNA library comprises at most 0.5% double stranded DNA from the original input DNA.

In an aspect of methods of nucleic acid analysis provided herein, the method may further comprise an enrichment step to enrich for one or more target sequences. In some cases, the method further comprises enriching the plurality of nucleic acid molecules for one or more target sequences. In some cases, enriching is performed with one or more primers or capture probes. In some cases, enriching is performed with one or more antibodies or fragments thereof.

In an aspect of nucleic acid analysis provided herein, identifying an individual nucleic acid molecule of a single-stranded DNA library as having a genomic feature; measuring a size for an individual nucleic acid molecule; or identifying a 5′ end or a 3′ end of an individual nucleic acid molecule may comprise sequencing a single stranded DNA library. In some cases, sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing.

In an aspect of nucleic acid analysis provided herein, methods may comprise identifying a nucleic acid molecule as having a genomic feature. In some cases, the genomic feature comprises an epigenetic modification. In some cases, the epigenetic modification is selected from the group consisting of methylation, phosphorylation, ubiquitination, sumoylation, acetylation, ribosylation, citrullination, and fragmentation. In some cases, the genomic feature comprises a copy number variation (CNV), a single nucleotide variant (SNV), an insertion, a deletion, a translocation, or a combination thereof.

In an aspect of nucleic acid analysis provided herein, a plurality of nucleic acid molecules may be derived from a sample, such as a cell-free biological sample. In some cases, the plurality of nucleic acid molecules is derived from a cell-free biological sample. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, a plurality of nucleic acids are derived from a subject suspected of having a disease, such as cancer. In some cases, a plurality of nucleic acids are derived from a healthy control. In some cases, the first plurality of nucleic acids comprises tumor nucleic acids. In some cases, the second plurality of nucleic acids is derived from a healthy control.

In an aspect of nucleic acid analysis provided herein, the analysis further provides risk of a disease or the presence of a disease in a subject. In some cases, the subject is determined to be at risk of or to have a disease when the individual nucleic acid molecule has an average size below a predetermined threshold. In some cases, the individual nucleic acid molecule having the genomic feature is determined to be associated with a disease when the individual nucleic acid molecule has an average size below a predetermined threshold. In some cases, the individual nucleic acid molecule having the genomic feature is determined to be associated with disease when the individual nucleic acid molecule having the genomic feature has an average size smaller than a nucleic acid molecule without the genomic feature. In some cases, the subject is determined to be at risk of or to have a disease when the individual nucleic acid molecule having the genomic feature has a size below a predetermined threshold. In some cases, the subject is determined to be at risk of or to have a disease when the 5′ end or the 3′ end of the individual nucleic acid molecule having the genomic feature has a predetermined sequence. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some cases, the method further comprises administering a therapeutic to the subject. In some cases, the method further comprises recommending additional cancer monitoring to the subject. In some cases, the method further comprises using the genomic feature, the size, or the 5′ end or the 3′ end to monitor the subject for a progression or a regression of the disease.

### Methods of Nucleic Acid Enrichment

In an aspect, there are provided methods for preparing a library enriched for cancer-derived nucleic acids. A method for preparing the library may comprise subjecting a population of nucleic acid molecules from a cell-free biological sample to enrichment nucleic acid molecules having sizes less than a predetermined threshold, thereby creating a library enriched for cancer-derived nucleic acids. In some cases, the predetermined threshold is 360 bp. In some cases, the predetermined threshold is 180 bp. In some cases, the predetermined threshold is 150 bp. In some cases, the predetermined threshold is 120 bp. In some cases, the predetermined threshold is 110 bp. In some cases, the predetermined threshold is 100 bp. In some cases, the predetermined threshold is 90 bp. In some cases, the predetermined threshold is 80 bp.

In aspects of methods of preparing a library herein, the enrichment may size selection of the population of nucleic acid molecules. In some cases, size selection comprises a bead purification or a gel purification of the population of nucleic acid molecules. In some cases, size selection comprises electrophoresis, capillary electrophoresis, or high-performance liquid chromatography.

Alternatively or in combination, in methods of preparing a library herein, the enrichment may comprise first ligating adapters to the population of nucleic acid molecules and then contacting the population of nucleic acid molecules with a cleaving agent. In some cases, the cleaving agent comprises a nuclease. In some cases, the cleaving agent comprises a chemical. In some cases, the cleaving agent comprises an acid. In some cases, the cleaving comprises bisulfite treatment. In some cases, the cleaving comprises sonication. In some cases, the cleaving comprises physical shearing. Additional non-limiting non-enzymatic cleaving methods are described in An, R. et al., 2014. Non-Enzymatic Depurination of Nucleic Acids: Factors and Mechanisms. PLoS ONE 9, e115950. In some cases, the enrichment comprises circularizing individual nucleic acid molecules of the population of nucleic acid molecules using an enzyme that favors small fragments.

Alternatively or in combination, in methods of preparing a library herein, the enrichment may comprise circularizing individual nucleic acid molecules of the population of nucleic acid molecules; and then contacting the population of nucleic acid molecules with a cleaving agent. In some cases, circularizing comprises ligating 5′ ends of the individual nucleic acid molecules to 3′ ends of the individual nucleic acid molecules. In some cases, circularizing comprises coupling adapters to 3′ ends, 5′ ends, or both 5′ ends and 3′ ends of the individual nucleic acid molecules.

In some aspects of methods of preparing a library herein, the method may comprise amplifying the nucleic acid molecules having sizes less than the predetermined threshold. In some cases, the amplifying is performed by a polymerase having strand displacement activity. In some cases, the amplifying is performed by a polymerase that does not have strand displacement activity. In some cases, the amplifying comprises contacting the individual nucleic acid molecules to an amplification reaction mixture comprising random primers. In some cases, the amplifying comprises contacting the individual nucleic acid molecules to an amplification reaction mixture comprising target-specific primers.

In some aspects of methods of preparing a library herein, the method may comprise enriching the nucleic acid molecules having sizes less than the predetermined threshold for one or more target sequences. In some cases, the enriching is performed with one or more primers or capture probes. In some cases, the enriching is performed with one or more antibodies or fragments thereof.

In an aspect of preparing a library provided herein, a plurality of nucleic acid molecules may be derived from a sample, such as a cell-free biological sample. In some cases, the plurality of nucleic acid molecules is derived from a cell-free biological sample. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, a plurality of nucleic acids are derived from a subject suspected of having a disease, such as cancer. In some cases, a plurality of nucleic acids are derived from a healthy control. In some cases, the first plurality of nucleic acids comprises tumor nucleic acids. In some cases, the second plurality of nucleic acids is derived from a healthy control.

### Methods of Amplification and Library Preparation

Methods herein may comprise amplification of polynucleotides present in a sample from a subject. Methods of amplification used herein may comprise rolling-circle amplification. Alternatively or in combination, methods of amplification used herein may comprise PCR. In some cases, methods of amplification herein comprise linear amplification. In some cases, amplification is not targeted to one gene or set of genes and the entire nucleic acid sample is amplified. In some cases, the method comprises circularizing individual polynucleotides of the plurality to form a plurality of circular polynucleotides, each of which having a junction between the 5′ end and the 3′ end and amplifying the circular polynucleotides of to produce amplified polynucleotides. In additional cases, methods of amplification comprise shearing the amplified polynucleotides to produce sheared polynucleotides, each sheared polynucleotide comprising one or more shear points at a 5′ end and/or 3′ end. In some cases, the method comprises enriching for a target sequence or a plurality of target sequences. In some cases, the method does not comprise enriching for a target sequence. In some cases, the method does not comprise aligning or mapping a cfDNA polynucleotide sequence to a reference genome. In some cases, the method does not comprise end repair.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”).

In some embodiments, samples comprise polynucleotides having a mixture of ends formed by natural degradation processes (such as cell lysis, cell death, and other processes by which polynucleotides such as DNA and RNA are released from a cell to its surrounding environment in which it may be further degraded, e.g., cell-free polynucleotides, e.g., cell-free DNA and cell-free RNA). Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide.

In some embodiments, circularizing individual polynucleotides is accomplished by subjecting the plurality of polynucleotides to a ligation reaction. The ligation reaction may comprise a ligase enzyme. In some embodiments, the ligase enzyme is degraded prior to amplifying. Degradation of ligase prior to amplifying can increase the recovery rate of amplifiable polynucleotides. In some embodiments, the plurality of circularized polynucleotides is not purified or isolated prior to amplification. In some embodiments, uncircularized, linear polynucleotides are degraded prior to amplifying.

Polynucleotides (e.g., polynucleotides from a sample) may be enriched prior to circularization. This may be performed using target specific primers. Alternatively, this may be performed using capture sequences, such as pull-down probes or capture sequences attached to a substrate (e.g., pull-down probes or capture sequences attached to an array or beads). Bait sets may be used to enrich for target-specific sequences before circularization.

In some cases, circularizing in comprises the operation of joining and adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

The circularized polynucleotides can be amplified, for example, after degradation of the ligase enzyme, to yield amplified polynucleotides. Amplifying the circular polynucleotides can be accomplished by a polymerase. In some cases, the polymerase is a polymerase having strand-displacement activity. In some cases, the polymerase is a Phi29 DNA polymerase. Alternatively, the polymerase is a polymerase that does not have strand-displacement activity. In some cases, the polymerase is a T4 DNA polymerase or a T7 DNA polymerase. Alternately or in combination, the polymerase is a Taq polymerase, or polymerase in the Taq polymerase family. In some cases, amplification comprises rolling circle amplification (RCA). The amplified polynucleotides resulting from RCA can comprise linear concatemers, or polynucleotides comprising more than one copy of a target sequence (e.g., subunit sequence) from a template polynucleotide. In some embodiments, amplifying comprises subjecting the circular polynucleotides to an amplification reaction mixture comprising random primers. In some embodiments, amplifying comprises subjecting the circular polynucleotides to an amplification reaction mixture comprising targeted primers. Alternatively, the circular polynucleotides may be amplified in an untargeted manner and enriched for one or more target sequences after amplification. In some cases, amplifying comprises subjecting the circular polynucleotides to an amplification reaction mixture comprising one or more primers, each of which specifically hybridizes to a different target sequence via sequence complementarity. In some cases, amplifying comprises subjecting the circular polynucleotides to an amplification reaction mixture comprising inverse primers.

Cell-free polynucleotides from a sample may be any of a variety of polynucleotides, including but not limited to, DNA, RNA, ribosomal RNA (rRNA), transfer RNA (tRNA), micro RNA (miRNA), small RNA, messenger RNA (mRNA), fragments of any of these, or combinations of any two or more of these. In some embodiments, samples comprise DNA. In some embodiments, samples comprise cell-free genomic DNA. In some embodiments, the samples comprise DNA generated by amplification, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, such as cell-free DNA and cell-free RNA, which may correspond to dead or damaged cells. The identity of such cells may be used to characterize the cells or population of cells from which they are derived, such as tumor cells (e.g. in cancer detection), fetal cells (e.g. in prenatal diagnostic), cells from transplanted tissue (e.g. in early detection of transplant failure), or members of a microbial community.

Where desired, polynucleotides from a sample may be fragmented prior to further processing. In some cases, fragmentation favors larger fragments therefore, smaller fragments of cell-free DNA may be enriched. Fragmentation may be accomplished by any of a variety of methods, including chemical, enzymatic, and mechanical fragmentation. In some embodiments, the fragments have an average or median length from about 10 to about 1,000 nucleotides in length, such as between 10-800, 10-500, 50-500, 90-200, or 50-150 nucleotides. In some embodiments, the fragments have an average or median length of about or less than about 100, 200, 300, 500, 600, 800, 1000, or 1500 nucleotides. In some embodiments, the fragments range from about 90-200 nucleotides, and/or have an average length of about 150 nucleotides. In some embodiments, the fragmentation is accomplished mechanically comprising subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg++ and in the presence of Mn++. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. Fragmented polynucleotides may be subjected to a step of size selecting the fragments via standard methods such as column purification, bead purification, or isolation from an agarose gel.

In some cases, methods herein comprise preparation of a DNA library from polynucleotides. For example, methods herein comprise preparation of a single stranded DNA library. Any suitable method of preparing a single stranded DNA library is contemplated for use in methods herein. For example, the method of preparing a single stranded DNA library comprises denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation comprises denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer.

Further examples of single stranded library preparation include a method comprising the steps of treating the DNA with a heat labile phosphatase to remove residual phosphate groups from the 5′ and 3′ ends of the DNA strands; removal of deoxyuracils derived from cytosine deamination from the DNA strands; ligation of a 5′-phosphorylated adapter oligonucleotide having about 10 nucleotides and a long 3′ biotinylated spacer arm to the 3′ ends of the DNA strands; immobilization of adapter-ligated molecules on streptavidin beads; copying the template strand using a 5′-tailed primer complementary to the adapter using Bst polymerase; washing away excess primers; removal of 3′ overhangs using T4 DNA polymerase; joining a second adapter to the newly synthesized strands using blunt-end ligation; washing away excess adapter; releasing library molecules by heat denaturation; adding full-length adapter sequences including bar codes through amplification using tailed primers; and sequencing the library, as described in Gansauge et al. 2013. Nature Protocols. 8(4) 737-748, which is entirely incorporated herein by reference.

In some library preparation approaches provided herein, certain nucleic acid molecules (e.g., cfDNA polynucleotides) are selected or enriched from a plurality of nucleic acid molecules (e.g., total cfDNA). Certain nucleic acid molecules or target sequences may be selected or enriched when they are more likely to result in informative results. For example, certain nucleic acid molecules or target sequences may be selected when they correspond to cfDNA sequences having altered size differences in subjects who have cancer (e.g., early stage cancer) as compared to healthy subjects. Certain nucleic acid molecules may be selected or enriched by amplification with target specific primers. Certain nucleic acid molecules may be selected or enriched by binding target nucleic acid molecules to probes. For example, such nucleic acid molecules are selected or enriched using bait sets.

In additional library preparation methods, cfDNA fragments having certain features are selected using an antibody. In some cases, cfDNA fragments that are methylated or hypermethylated are selected using an antibody. Selected cfDNA fragments are then used in any library preparation method described herein, including circularization, single stranded DNA library preparation, and double stranded DNA library preparation. Sequencing such isolated cfDNA fragments provides information as to the features present in the cfDNA, including modifications such as methylation or hypermethylation.

According to some embodiments, polynucleotides among the plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). In some embodiment, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, the concentration of various reagents, and the concentration of polynucleotides to be joined. In some embodiments, a circularization reaction preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

In some cases, rather than preferentially forming self-joining circularization products, one or more adapter oligonucleotides are used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide. Adapter oligonucleotides can comprise DNA, RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. When two hybridized regions of an adapter are separated from one another by a non-hybridized region, a “bubble” structure results. Adapters of different kinds can be used in combination, such as adapters of different sequences. Different adapters can be joined to sample polynucleotides in sequential reactions or simultaneously. In some embodiments, identical adapters are added to both ends of a target polynucleotide. For example, first and second adapters can be added to the same reaction. Adapters can be manipulated prior to combining with sample polynucleotides. For example, terminal phosphates can be added or removed.

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In some cases, the adapters may be used to purify those circles that contain the adapters, for example by using beads (particularly magnetic beads for ease of handling) that are coated with oligonucleotides comprising a complementary sequence to the adapter, that can “capture” the closed circles with the correct adapters by hybridization thereto, wash away those circles that do not contain the adapters and any unligated components, and then release the captured circles from the beads. In addition, in some cases, the complex of the hybridized capture probe and the target circle can be directly used to generate concatemers, such as by direct rolling circle amplification (RCA). In some embodiments, the adapters in the circles can also be used as a sequencing primer. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. A sequence element may be of any suitable length, such as about or less than about 3, 4, 5, 6, 7, 8, 9, 10, 15, 20, 25, 30, 35, 40, 45, 50 or more nucleotides in length. Adapter oligonucleotides can have any suitable length, at least sufficient to accommodate the one or more sequence elements of which they are comprised. In some embodiments, adapters are about or less than about 10, 15, 20, 25, 30, 35, 40, 45, 50, 55, 60, 65, 70, 75, 80, 90, 100, 200, or more nucleotides in length. In some embodiments, an adapter oligonucleotide is in the range of about 12 to 40 nucleotides in length, such as about 15 to 35 nucleotides in length.

In some embodiments, the adapter oligonucleotides joined to fragmented polynucleotides from one sample comprise one or more sequences common to all adapter oligonucleotides and a barcode that is unique to the adapters joined to polynucleotides of that particular sample, such that the barcode sequence can be used to distinguish polynucleotides originating from one sample or adapter joining reaction from polynucleotides originating from another sample or adapter joining reaction. In some embodiments, an adapter oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an adapter oligonucleotide may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

A variety of methods for circularizing polynucleotides are available. In some embodiments, circularization comprises an enzymatic reaction, such as use of a ligase (e.g. an RNA or DNA ligase). A variety of ligases are available, including, but not limited to, Circligase™ (Epicentre; Madison, Wis.), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. Where self-joining is desired, the concentration of polynucleotides and enzyme can be adjusted to facilitate the formation of intramolecular circles rather than intermolecular structures. Reaction temperatures and times can be adjusted as well. In some embodiments, 60° C. is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides, such as cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood that two polynucleotides will have the same 5′ end or 3′ end is low, and the likelihood that two polynucleotides will independently have both the same 5′ end and 3′ end is extremely low. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

### Methods of Sequencing

According to some embodiments, linear and/or circularized polynucleotides (or amplification products thereof, which may have been enriched in some cases) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, MGI, etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the α and β phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891, which is entirely incorporated herein by reference.

In related sequencing processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex, allowing identification of the incorporated nucleotide. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes. For example, the Illumina Genome Analyzer System is based on technology described in WO 98/44151, wherein DNA molecules are bound to a sequencing platform (flow cell) via an anchor probe binding site (otherwise referred to as a flow cell binding site) and amplified in situ on a glass slide. A solid surface on which DNA molecules are amplified may comprise a plurality of first and second bound oligonucleotides, the first complementary to a sequence near or at one end of a target polynucleotide and the second complementary to a sequence near or at the other end of a target polynucleotide. This arrangement permits bridge amplification, such as described in US20140121116. The DNA molecules are then annealed to a sequencing primer and sequenced in parallel base-by-base using a reversible terminator approach. Hybridization of a sequencing primer may be preceded by cleavage of one strand of a double-stranded bridge polynucleotide at a cleavage site in one of the bound oligonucleotides anchoring the bridge, thus leaving one single strand not bound to the solid substrate that may be removed by denaturing, and the other strand bound and available for hybridization to a sequencing primer. In some cases, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β,γ, or other terminal phosphate group on a nucleoside polyphosphate, the label is not incorporated into the nascent strand, and instead, natural DNA is produced. Observation of individual molecules may involve the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,917,726, 7,033,764, 7,052,847, 7,056,676, 7,170,050, 7,361,466, and 7,416,844; and US 20070134128, each of which is entirely incorporated herein by reference).

In some embodiments, the nucleic acids in the sample can be sequenced by ligation. This method may use a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

In some embodiments, the nucleic acids in the sample are sequenced using nanopore technology.

Sequencing methods herein provide information useful in methods herein. In some cases, sequencing provides a sequence of a polymorphic region. Additionally, sequencing provides a length of a polynucleotide, such as a DNA including cfDNA. Further, sequencing provides a sequence of a breakpoint or end of a DNA such as a cfDNA. Sequencing further provides a sequence of a border of a protein binding site or a border of a DNase hypersensitive site.

### Computer Systems

The present disclosure provides computer systems that are programmed to implement methods of the disclosure. FIG. 7 shows a computer system 701 that is programmed or otherwise configured to implement methods of the present disclosure. The computer system 701 can regulate various aspects of methods of the present disclosure, such as, for example, methods for determining that a subject has or is at risk of having a disease (e.g., cancer).

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a healthcare provider or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, results of methods of the present disclosure. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can be, for example, a trained algorithm (or trained machine learning algorithm), such as, for example, a support vector machine or neural network.

### Samples

In embodiments of the various methods described herein, the sample may be from a subject. A subject may be any animal, including but not limited to, a cow, a pig, a mouse, a rat, a chicken, a cat, a dog, etc., and is usually a mammal, such as a human. Sample polynucleotides may be isolated from a subject, such as a tissue sample, bodily fluid sample, or organ sample, including, for example, biopsy, blood sample, or fluid sample containing nucleic acids (e.g. saliva). In some cases, the sample does not comprise intact cells, is treated to remove cells, or polynucleotides are isolated without a cellular extractions step (e.g. to isolate cell-free polynucleotides, such as cell-free DNA). Other examples of sample sources include those from blood, urine, feces, nares, the lungs, the gut, other bodily fluids or excretions, materials derived therefrom, or combinations thereof. In some embodiments, the sample is a blood sample or a portion thereof (e.g. blood plasma or serum). Serum and plasma may be of particular interest, due to the relative enrichment for tumor DNA associated with the higher rate of malignant cell death among such tissues. In some embodiments, a sample from a single individual is divided into multiple separate samples (e.g. 2, 3, 4, 5, 6, 7, 8, 9, 10, or more separate samples) that are subjected to methods of the disclosure independently, such as analysis in duplicate, triplicate, quadruplicate, or more. Where a sample is from a subject, the reference sequence may also be derived of the subject, such as a consensus sequence from the sample under analysis or the sequence of polynucleotides from another sample or tissue of the same subject. For example, a blood sample may be analyzed for ctDNA mutations, while cellular DNA from another sample (e.g. buccal or skin sample) is analyzed to determine the reference sequence.

Polynucleotides may be extracted from a sample according to any suitable method. A variety of kits are available for extraction of polynucleotides, selection of which may depend on the type of sample, or the type of nucleic acid to be isolated. Examples of extraction methods are provided herein, such as those described with respect to any of the various aspects disclosed herein. In one example, the sample may be a blood sample, such as a sample collected in an EDTA tube (e.g. BD Vacutainer). Plasma can be separated from the peripheral blood cells by centrifugation (e.g. 10 minutes at 1900×g at 4° C.). Plasma separation performed in this way on a 6 mL blood sample may yield 2.5 to 3 mL of plasma. Circulating cell-free DNA can be extracted from a plasma sample, such as by using a QIAmp Circulating Nucleic Acid Kit (Qiagene), according the manufacturer's protocol. DNA may then be quantified (e.g. on an Agilent 2100 Bioanalyzer with High Sensitivity DNA kit (Agilent)). As an example, yield of circulating DNA from such a plasma sample from a healthy person may range from 1 ng to 10 ng per mL of plasma, with significantly more in cancer patient samples.

In some embodiments, the plurality of polynucleotides comprises cell-free polynucleotides, such as cell-free DNA (cfDNA), cell-free RNA (cfRNA), circulating tumor DNA (ctDNA), or circulating tumor RNA (ctRNA). Cell-free DNA circulates in both healthy and diseased individuals. Cell-free RNA circulates in both healthy and diseased individuals. cfDNA from tumors (ctDNA) is not confined to any specific cancer type, but appears to be a common finding across different malignancies. According to some measurements, the free circulating DNA concentration in plasma is about 14-18 ng/ml in control subjects and about 180-318 ng/ml in patients with neoplasias. Apoptotic and necrotic cell death contribute to cell-free circulating DNA in bodily fluids. For example, significantly increased circulating DNA levels have been observed in plasma of prostate cancer patients and other prostate diseases, such as Benign Prostate Hyperplasia and Prostatits. In addition, circulating tumor DNA is present in fluids originating from the organs where the primary tumor occurs. Thus, breast cancer detection can be achieved in ductal lavages; colorectal cancer detection in stool; lung cancer detection in sputum, and prostate cancer detection in urine or ejaculate. Cell-free DNA may be obtained from a variety of sources. One common source is blood samples of a subject. However, cfDNA or other fragmented DNA may be derived from a variety of other sources. For example, urine and stool samples can be a source of cfDNA, including ctDNA. Cell-free RNA may be obtained from a variety of sources.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides. For example, cell-free polynucleotides may include cell-free DNA (also called “circulating” DNA). In some embodiments, the circulating DNA is circulating tumor DNA (ctDNA) from tumor cells, such as from a body fluid or excretion (e.g. blood sample). Cell-free polynucleotides may include cell-free RNA (also called “circulating” RNA). In some embodiments, the circulating RNA is circulating tumor RNA (ctRNA) from tumor cells. Tumors frequently show apoptosis or necrosis, such that tumor nucleic acids are released into the body, including the blood stream of a subject, through a variety of mechanisms, in different forms and at different levels. In some cases, the size of the ctDNA can range between higher concentrations of smaller fragments, generally 70 to 200 nucleotides in length, to lower concentrations of large fragments of up to thousands kilobases.

### Cancer

Methods herein may provide for detection of cancer, for example, in some cases, early stage cancer can be detected. Staging of cancer may be dependent on cancer type where each cancer type has its own classification system. Examples of cancer staging or classification systems are described in more detail below.

TABLE 1
Colon Cancer Primary Tumor (T)

| | |
|---|---|
| TX | Primary tumor cannot be assessed |
| T0 | No evidence of primary tumor |
| Tis | Carcinoma in situ: intraepithelial or intramucosal carcinoma (involvement of lamina propria with |
| | no extension through the muscularis mucosa) |
| T1 | Tumor invades submucosa (through the muscularis mucosa but not into the muscularis propria) |
| T2 | Tumor invades muscularis propria |
| T3 | Tumor invades through the muscularis propria into the pericolorectal tissues |
| T4 | Tumor invades the visceral peritoneum or invades or adheres to adjacent organ or structure |
| T4a | Tumor invades through the visceral peritoneum (including gross perforation of the bowel |
| | through tumor and continuous invasion of tumor through areas of inflammation to the surface of |
| | the visceral peritoneum) |
| T4b | Tumor directly invades or is adherent to other organs or structures |

Colon Cancer Regional Lymph Notes (N)

| | |
|---|---|
| NX | Regional lymph nodes cannot be assessed |
| N0 | No regional lymph node metastasis |
| N1 | Metastasis in 1-3 regional lymph nodes (tumor in lymph nodes measuring ≥0.2 mm) or any |
| | number of tumor deposits are present and all identifiable nodes are negative |
| N1a | Metastasis in 1 regional lymph node |
| N1b | Metastasis in 2-3 regional lymph nodes |
| N1c | Tumor deposit(s) in the subserosa, mesentery, or nonperitonealized, pericolic, or perirectal/ |
| | mesorectal tissues without regional nodal metastasis |
| N2 | Metastasis in 4 or more lymph nodes |
| N2a | Metastasis in 4-6 regional lymph nodes |
| N2b | Metastasis in 7 or more regional lymph nodes |

Colon Cancer Distant Metastasis (M)

| | |
|---|---|
| M0 | No distant metastasis by imaging or other studies, no evidence of tumor in distant sites or organs. |
| | (This category is not assigned by pathologists.) |
| M1 | Metastasis to one or more distant sites or organs or peritoneal metastasis |
| M1a | Metastasis confined to 1 organ or site (e.g., liver, lung, ovary, nonregional node) without |
| | peritoneal metastasis |
| M1b | Metastasis to two or more sites or organs without peritoneal metastasis |
| M1c | Metastasis to the peritoneal surface alone or with other site or organ metastases |

TABLE 2
Colon Cancer Anatomic stage/prognostic groups

| Stage | T | N | M | Dukes | MAC |
|---|---|---|---|---|---|
| 0 | Tis | N0 | M0 | — | — |
| I | T1 | N0 | M0 | A | A |
| | T2 | N0 | M0 | A | B1 |
| IIA | T3 | N0 | M0 | B | B2 |
| IIB | T4a | N0 | M0 | B | B2 |
| IIC | T4b | N0 | M0 | B | B3 |
| IIIA | T1-T2 | N1/N1c | M0 | C | C1 |
| | T1 | N2a | M0 | C | C1 |
| IIIB | T3-T4a | N1/N1c | M0 | C | C2 |
| | T2-T3 | N2a | M0 | C | C1/C2 |
| | T1-T2 | N2b | M0 | C | C1 |
| IIIC | T4a | N2a | M0 | C | C2 |
| | T3-T4a | N2b | M0 | C | C2 |
| | T4b | N1-N2 | M0 | C | C3 |
| IVA | Any T | Any N | M1a | — | — |
| IVB | Any T | Any N | M1b | — | — |
| IVC | Any T | Any N | M1c | — | — |

TABLE 3
Malignant Melanoma Primary Tumor (T)

| | |
|---|---|
| TX | Primary tumor cannot be assessed (i.e. curettaged melanoma) |
| T0 | No evidence of primary tumor |
| Tis | Melanoma in situ |
| T1 | Thickness ≤1.0 mm |
| | T1a: <0.8 mm without ulceration |
| | T1b: <0.8 mm with ulceration, or 0.8-1.0 mm with or without ulceration |
| T2 | Thickness >1.0-2.0 mm |
| | T2a: Without ulceration |
| | T2b: With ulceration |
| T3 | Thickness >2.0-4.0 mm |
| | T3a: Without ulceration |
| | T3b: With ulceration |
| T4 | Thickness >4.0 mm |
| | T4a: Without ulceration |
| | T4b: With ulceration |

Malignant Melanoma Regional Lymph Notes (N)

| | |
|---|---|
| NX | Regional lymph nodes cannot be assessed |
| N0 | No regional metastasis detected |
| N1 | One tumor-involved lymph node or in-transit, satellite, and/or microsatellite metastases with no |
| | tumor-involved nodes |
| | N1a: One clinically occult (i.e., detected by sentinel lymph node biopsy [SLNB]; no in-transit, |
| | satellite, or microsatellite metastases |
| | N1b: One clinically detected; no in-transit, satellite, or microsatellite metastases |
| | N1c: No regional lymph node disease; in-transit, satellite, and/or microsatellite metastases found |
| N2 | Two or three tumor-involved nodes; or in-transit, satellite, or microsatellite metastases |
| | N2a: Two or three clinically occult (i.e., detected by SLNB); no in-transit, satellite, or |
| | microsatellite metastases |
| | N2b: Two or three clinically detected; no in-transit, satellite, or microsatellite metastases |
| | N2c: One clinically occult or clinically detected; in-transit, satellite, and/or microsatellite |
| | metastases found |
| N3 | ≥4 tumor-involved nodes or in -transit, satellite, and/or microsatellite metastases with ≥2 tumor- |
| | involved nodes or any number of matted nodes without or with in-transit, satellite, and/or |
| | microsatellite metastases |
| | N3a: ≥4 clinically occult (i.e., detected by SLNB); no in-transit, satellite, or microsatellite |
| | metastases |
| | N3b: ≥4, at least one of which was clinically detected, or presence of any matted nodes; no in- |
| | transit, satellite, or microsatellite metastases |
| | N3c: ≥2 clinically occult or clinically detected and/or presence of any matted nodes, with |
| | presence of in-transit, satellite, and/or microsatellite metastases |

Malignant Melanoma Distant Metastasis (M)

| | |
|---|---|
| M0 | No detectable evidence of distant metastases |
| M1a | Metastases to skin, soft tissue (including muscle), and/or nonregional lymph nodes |
| M1b | Lung metastasis, with or without M1a involvement |
| M1c | Distant metastasis to non-central nervous system (CNS) visceral sites with or without M1a or |
| | M1b involvement |
| M1d | Distant metastasis to CNS, with or without M1a or M1b involvement |

TABLE 4
Malignant Melanoma Anatomic stage/prognostic groups

| Stage | T | N | M |
|---|---|---|---|
| 0 | Tis | N0 | M0 |
| IA | T1a | N0 | M0 |
| IB | T1b | N0 | M0 |
| | T2a | N0 | M0 |
| IIA | T2b | N0 | M0 |
| | T3a | N0 | M0 |
| IIB | T3b | N0 | M0 |
| | T4a | N0 | M0 |
| IIC | T4b | N0 | M0 |
| III | Any T, Tis | N1, N2, or N3 | M0 |
| IV | Any T | Any N | M1 |

TABLE 5
Hepatocellular Carcinoma Primary tumor (T)

| | |
|---|---|
| TX | Primary tumor cannot be assessed |
| T0 | No evidence of primary tumor |
| T1 | Solitary tumor 2 cm without vascular invasion |
| T1a | Solitary tumor <2 cm |
| T1b | Solitary tumor >2 cm without vascular invasion |
| T2 | Solitary tumor >2 cm with vascular invasion; or multiple tumors, non >5 cm |
| T3 | Multiple tumors, at least one of which is >5 cm |
| T4 | Single tumor or tumors of any size involving a major branch of the portal vein or |
| | hepatic vein, or tumor(s) with direct invasion of adjacent organs other than the |
| | gallbladder or with perforation of visceral peritoneum |

Hepatocellular Carcinoma Regional Lymph Nodes (N)

| | |
|---|---|
| NX | Regional lymph node(s) cannot be assessed |
| N0 | No regional lymph node metastasis |
| N1 | Regional lymph node metastasis |

Hepatocellular Carcinoma Distant Metastasis (M)

| | |
|---|---|
| M0 | No distant metastasis |
| M1 | Distant metastasis |

TABLE 6
Hepatocellular Carcinoma Anatomic stage/prognostic groups

| Stage | T | N | M |
|---|---|---|---|
| IA | T1a | N0 | M0 |
| IB | T1b | N0 | M0 |
| II | T2 | N0 | M0 |
| IIIA | T3 | N0 | M0 |
| IIIB | T4 | N0 | M0 |
| IVA | Any T | N1 | M0 |
| IVB | Any T | Any N | M1 |

TABLE 7
Hepatocellular Carcinoma Histologic grade

| | |
|---|---|
| GX | Grade cannot be accessed |
| G1 | Well differentiated |
| G2 | Moderately differentiated |
| G3 | Poorly differentiated |
| G4 | Undifferentiated |

TABLE 8
Barcelona-Clinic Liver Cancer staging system

| | Performance | | Okuda | |
|---|---|---|---|---|
| Stage | Status | Tumor Stage | Stage | Liver function |
| A: Early HCC | | | | |
| A1 | 0 | Single, <5 cm | I | No portal |
| | | | | hypertension, normal |
| | | | | bilirubin |
| A2 | 0 | Single, <5 cm | I | Portal hypertension, |
| | | | | normal bilirubin |
| A3 | 0 | Single, <5 cm | I | Portal hypertension, |
| | | | | normal bilirubin |
| A4 | 0 | 3 tumors, <3 cm | I-II | Child-Pugh A-B |
| Stage B: Intermediate | 0 | Large, | I-II | Child-Pugh A-B |
| HCC | | multinodular | | |
| Stage C: Advanced | 1-2 | Vascular invasion | I-II | Child-Pugh A-B |
| HCC | | or extrahepatic | | |
| | | spread | | |
| Stage D: End-Stage | 3-4 | Any | I-II | Child-Pugh C |
| HCC | | | | |

TABLE 9
Ishak Fibrosis score

| Architectural Change | Score |
|---|---|
| No fibrosis | 0 |
| Fibrous expansion of some portal areas, with or | 1 |
| without short fibrous septa | |
| Fibrous expansion of most portal areas, with or | 2 |
| without short fibrous septa | |
| Fibrous expansion of portal areas with occasional | 3 |
| portal-to-portal bridging | |
| Fibrous expansion of portal areas with marked | 4 |
| bridging as well as portal-central | |
| Marked bridging (portal-to-portal and/or portal- | 5 |
| central) with occasional nodule (incomplete | |
| cirrhosis) | |
| Cirrhosis, probable or definite | 6 |

TABLE 10
Gastric Cancer Primary tumor (T)

| | |
|---|---|
| TX | Primary tumor cannot be assessed |
| T0 | No evidence of primary tumor |
| Tis | Carcinoma in situ: intraepithelial tumor without invasion of the lamina propria |
| T1 | Tumor invades lamina propria, muscularis mucosae, or submucosa |
| T1a | Tumor invades lamina propria or muscularis mucosae |
| T1b | Tumor invades submucosa |
| T2 | Tumor invades muscularis propria |
| T3 | Tumor penetrates subserosal connective tissue without invasion of visceral peritoneum |
| | or adjacent structures. |
| T4 | Tumor invades serosa (visceral peritoneum) or adjacent structures |
| T4a | Tumor invades serosa (visceral peritoneum) |
| T4b | Tumor invades adjacent structures |

Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in seven or more regional lymph nodes |
| N3a | Metastasis in 7-15 regional lymph nodes |
| N3b | Metastasis in 16 or more regional lymph nodes |

Distant Metastasis (M)

| | |
|---|---|
| M0 | No distant metastasis |
| M1 | Distant metastasis |

TABLE 11
Gastric Cancer Clinical stage/prognostic groups (cTNM)

| Stage | T | N | M |
|---|---|---|---|
| 0 | Tis | N0 | M0 |
| I | T1 | N0 | M0 |
| | T2 | N0 | M0 |
| IIA | T1 | N1, N2, N3 | M0 |
| | T2 | N1, N2, N3 | M0 |
| IIB | T3 | N0 | M0 |
| | T4 | N0 | M0 |
| III | T | N1, N2, N3 | M0 |
| | T4a | N1, N2, N3 | M0 |
| IVA | Any T | Any N | M0 |
| IVB | Any T | Any N | M1 |

TABLE 12
Gastric Cancer Pathological stage (pTNM)

| Stage | T | N | M |
|---|---|---|---|
| 0 | Tis | N0 | M0 |
| I | T1 | N0 | M0 |
| | T1 | N1 | M0 |
| IB | T2 | N0 | M0 |
| | T1 | N2 | M0 |
| II A | T2 | N1 | M0 |
| | T3 | N0 | M0 |
| | T1 | N3 | M0 |
| | T2 | N2 | M0 |

TABLE 13
Gastric Cancer Post-neoadjuvant therapy
staging and overall survival (ypTNM)

| | | | | 3-year | 5-year |
|---|---|---|---|---|---|
| Stage | T | N | M | survival (%) | survival (%) |
| I | T1, T2 | N0 | M0 | 81.4 | 76.5 |
| | T1 | N1 | M0 | | |
| | T1 | N2, N3 | M0 | | |
| | T2 | N1, N2 | M0 | | |
| II | T3 | N0, N1 | M0 | 54.8 | 46.3 |
| | T4a | N0 | M0 | | |
| | T2 | N3 | M0 | | |
| | T3 | M2, N3 | M0 | | |
| III | T4a | N1, N2, N3 | M0 | | |
| | T4b | N0, N1, N2, N3 | M0 | 28.8 | 18.3 |
| IV | Any T | Any N | M1 | 10.2 | 5.7 |

TABLE 14
Esophageal Cancer Primary tumor (T)

| | |
|---|---|
| TX | Primary tumor cannot be assessed |
| T0 | No evidence of primary tumor |
| Tis | High-grade dysplasia,* defined as malignant cells confined by |
| | the basement membrane |
| T1 | Tumor invades lamina propria, muscularis mucosae, or submucosa |
| T1a | Tumor invades lamina propria or muscularis mucosae |
| T1b | Tumor invades submucosa |
| T2 | Tumor invades muscularis propria |
| T3 | Tumor invades adventitia |
| T4 | Tumor invades adjacent structures |
| T4a | Resectable tumor invading pleura, pericardium, azygos vein, |
| | diaphragm or peritoneum |
| T4b | Unresectable tumor invading other adjacent structures, such as |
| | the aorta, vertebral body, and trachea |

Esophageal Cancer Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in 7 or more regional lymph nodes |

Esophageal Cancer Distant Metastasis (M)

| | |
|---|---|
| M0 | No distant metastasis |
| M1 | Distant metastasis |

TABLE 15
Esophageal Cancer Histologic grade
Histologic grade (G)

| | |
|---|---|
| GX | Grade cannot be assessed - stage grouping as G1 |
| G1 | Well differentiated |
| G2 | Moderately differentiated |
| G3 | Poorly differentiated or undifferentiated* |

TABLE 16
Squamous cell carcinoma location

| | |
|---|---|
| X | Location unknown |
| Upper | Cervical esophagus to lower border of azygos vein |
| Middle | Lower border of azygos vein to lower border of |
| | inferior pulmonary vein |
| Lower | Lower border of inferior pulmonary vein to stomach, |
| | including gastroesophageal junction |

TABLE 17
Esophageal Cancer Clinical stage groups

| | | | |
|---|---|---|---|
| Stage Group | cT | cN | cM |

Squamous cell carcinoma

| | | | |
|---|---|---|---|
| 0 | Tis | N0 | M0 |
| I | T1 | N0-1 | M0 |
| | T2 | N0-1 | M0 |
| II | T3 | N0 | M0 |
| | T3 | N1 | M0 |
| III | T1-3 | N2 | M0 |
| | T4 | N0-2 | M0 |
| IVA | T1-4 | N3 | M0 |
| IVB | T1-4 | N0-3 | M1 |

Adenocarcinoma

| | | | |
|---|---|---|---|
| 0 | Tis | N0 | M0 |
| I | T1 | N0 | M0 |
| IIA | T1 | N1 | M0 |
| IIB | T2 | N0 | M0 |
| | T2 | N1 | M0 |
| III | T3-4a | N0-1 | M0 |
| | T1-4a | N2 | M0 |
| IVA | T4b | N0-2 | M0 |
| | T1-4 | N3 | M0 |
| IVB | T1-4 | N0-3 | M1 |

TABLE 18
Pathologic stage groups (Open Table in a new window)

| | | | | | |
|---|---|---|---|---|---|
| Stage Group | pT | pN | pM | Grade | Location |

Squamous cell carcinoma

| | | | | | |
|---|---|---|---|---|---|
| 0 | Tis | N0 | M0 | N/A | Any |
| IA | T1a | N0 | M0 | G-1, X | Any |
| | T1b | N0 | M0 | G1-3, X | Any |
| IB | T1a | N0 | M0 | G2-3 | Any |
| | T2 | N0 | M0 | G1 | Any |
| | T2 | N0 | M0 | G2-3, X | Any |
| IIA | T3 | N0 | M0 | Any | Lower |
| | T34 | N0 | M0 | G1 | Upper/middle |
| | T3 | N0 | M0 | G2-3 | Upper/middle |
| | T3 | N0 | M0 | GX | Any |
| IIB | T3 | N0 | M0 | Any | X |
| | T1 | N1 | M0 | Any | Any |
| IIIA | T1 | N2 | M0 | Any | Any |
| | T2 | N1 | M0 | Any | Any |
| | T4a | N0-1 | M0 | Any | Any |
| IIIB | T3 | N1 | M0 | Any | Any |
| | T2-3 | N2 | M0 | Any | Any |
| | T4a | N2 | M0 | Any | Any |
| IVA | T4b | N0-2 | M0 | Any | Any |
| | T1-4 | N3 | M0 | Any | Any |
| IVB | T1-4 | N0-3 | M1 | Any | Any |

Adenocarcinoma

| | | | | |
|---|---|---|---|---|
| 0 | Tis | N0 | M0 | N/A |
| IA | T1a | N0 | M0 | G1, X |
| IB | T1a | N0 | M0 | G2 |
| | T1b | N0 | M0 | G1-2, X |
| | T1 | N0 | M0 | G3 |
| IC | T2 | N0 | M0 | G1-2 |
| IIA | T2 | N0 | M0 | G3, X |
| | T1 | N1 | M0 | Any |
| IIB | T3 | N0 | M0 | Any |
| | T1 | N2 | M0 | Any |
| IIIA | T2 | N1 | M0 | Any |
| | T4a | N0-1 | M0 | Any |
| IIIB | T3 | N1 | M0 | Any |
| | T2-3 | N2 | M0 | Any |
| IVA | T4a | N2 | M0 | Any |
| | T4b | N0-2 | M0 | Any |
| | T1-4 | N3 | M0 | Any |
| | R1-4 | N0-3 | M1 | Any |

TABLE 19
Postneoadjuvant therapy staging (Open Table in a new window)

| | | | |
|---|---|---|---|
| Stage Group | ypT | ypN | ypM |

Squamous cell carcinoma

| | | | |
|---|---|---|---|
| I | T0-2 | N0 | M0 |
| II | T3 | N0 | M0 |
| IIIA | T0-2 | N1 | M0 |
| | T4a | N0 | M0 |
| IIIB | T3 | N1 | M0 |
| | T0-3 | N2 | M0 |
| | T4a | N1-2, X | M0 |
| IVA | T4b | N0-2 | M0 |
| | T1-4 | N3 | M0 |
| IVB | T1-4 | N0-3 | M1 |

TABLE 20

| | | |
|---|---|---|
| TNM | FIGO stages | Surgical-pathologic findings |

Endometrial Cancer Primary Tumor (T)

| | | |
|---|---|---|
| TX | | Primary tumor cannot be assessed |
| T0 | | No evidence of primary tumor |
| Tis | | Carcinoma in situ (preinvasive carcinoma) |
| T1 | I | Tumor confined to corpus uteri |
| T1a | IA | Tumor linked to endometrium or invades less than one half of |
| | | the myometrium |
| T1b | IB | Tumor invades one half or more of the myometrium |
| T2 | II | Tumor invades stromal connective tissue of the cervix but does |
| | | not extend beyond uterus** |
| T3a | IIIA | Tumor involves serosa and/or adnexa (direct extension or |
| | | metastasis) |
| T3b | IIIB | Vaginal involvement (direct extension or metastasis) or |
| | | parametrial involvement |
| | IIIC | Metastases to pelvic and/or para-aortic lymph nodes |
| | IV | Tumor invades bladder mucosa and/or bowel mucosa, and/or |
| | | distant metastases |
| T4 | IVA | Tumor invades bladder mucosa and/or bowel mucosa (bullous |
| | | edema is not sufficient to classify a tumor as T4) |

Endometrial Cancer Regional Lymph Nodes (N)

| | | |
|---|---|---|
| TNM | FIGO | Surgical-pathologic findings |
| | stages | |
| NX | | Regional lymph nodes cannot be assessed |
| N0 | | No regional lymph node metastasis |
| N1 | IIIC1 | Regional lymph node metastasis to pelvic lymph nodes |
| N2 | IIIC2 | Regional lymph node metastasis to para-aortic lymph nodes, |
| | | with or without positive pelvic lymph nodes |

Endometrial Cancer Distant Metastasis

| | | |
|---|---|---|
| TNM | FIGO | Surgical-pathologic findings |
| | stages | |
| M0 | | No distant metastasis |
| M1 | | Distant metastasis (includes metastasis to inguinal lymph nodes, |
| | | intraperitoneal M1 IVB disease, or lung, liver, or bone |
| | | metastases; it excludes metastasis to para-aortic lymph nodes, |
| | | vagina, pelvic serosa, or adnexa) |

TABLE 21
Non-Small Cell Lung Cancer Primary tumor (T)

| | |
|---|---|
| TX | Primary tumor cannot be assessed, or tumor is proven by the presence of malignant cells |
| | in sputum or bronchial washings but not visualized by imaging or bronchoscopy |
| T0 | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor >1 cm but ≤2 cm in greatest dimension |
| T1c | Tumor >2 cm but ≤3 cm in greatest dimension |
| T2 | Tumor >3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor >3 cm but ≤4 cm in greatest dimension |
| T2b | Tumor >4 cm but ≤5 cm in greatest dimension |
| T3 | Tumor >5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor >7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Non-Small Cell Lung Cancer Regional lymph nodes (N)

| | |
|---|---|
| NX | Regional lymph nodes cannot be assessed |
| N0 | No regional node metastasis |
| N1 | Metastasis in ipsilateral peribronchial and/or ipsilateral hilar lymph nodes and |
| | intrapulmonary nodes, including involvement by direct extension |
| N2 | Metastasis in ipsilateral mediastinal and/or subcarinal lymph node(s) |
| N3c | Metastasis in the contralateral mediastinal, contralateral hilar, ipsilateral or contralateral |
| | scalene, or supraclavicular lymph node(s) |

Non-Small Cell Lung Cancer Distant metastasis (M)

| | |
|---|---|
| M0 | No distant metastasis |
| M1 | Distant metastasis |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer are a result of the tumor. In a few patients, however, multiple microscopic |
| | examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging descriptor. |
| M1b | Single extrathoracic metastasis in a single organ and involvement of a single nonregional |
| | node |
| M1c | Multiple extrathoracic metastases in a single organ or in multiple organs |

TABLE 22
Non-Small Cell Lung Cancer Anatomic stage/prognostic groups

| Stage | T | N | M |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| | T2a | N2 | M0 |
| IIIA | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| | T3 | N3 | M0 |
| IIIC | T4 | N3 | M0 |
| IVA | T Any | N Any | M1a |
| T Any | | N Any | M1b |
| IVB | T Any | N Any | M1c |

TABLE 23
Small Cell Lung Cancer Primary tumor (T)

| | |
|---|---|
| TX | Primary tumor cannot be assessed, or tumor is proven by the presence of malignant cells in |
| | sputum or bronchial washings but not visualized by imaging or bronchoscopy |
| TC | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor >1 cm but ≤2 cm in greatest dimension |
| T1c | Tumor >2 cm but ≤3 cm in greatest dimension |
| T2 | Tumor >3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor >3 cm but ≤4 cm in greatest dimension |
| T2b | Tumor >4 cm but ≤5 cm in greatest dimension |
| T3 | Tumor >5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor >7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Small Cell Lung Cancer Regional lymph nodes (N)

| | |
|---|---|
| NX | Regional lymph nodes cannot be assessed |
| N0 | No regional lymph node metastasis |
| N1 | Metastasis to ipsilateral peribronchial and/or ipsilateral hilar lymph nodes and |
| | intrapulmonary nodes, including involvement by direct extension |
| N2 | Metastases in ipsilateral mediastinal and/or subcarinal lymph node(s) |
| N3 | Metastasis in contralateral mediastinal, contralateral hilar, ipsilateral or contralateral |
| | scalene, or supraclavicular lymph node(s) |

Small Cell Lung Cancer Distant metastasis (M)

| | |
|---|---|
| M0 | No distant metastasis |
| M1 | Distant metastases |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer are a result of the tumor. In a few patients, however, multiple microscopic |
| | examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging descriptor. |
| M1b | Single extrathoracic metastasis in a single organ and involvement of a single nonregional |
| | node |
| M1c | Multiple extrathoracic metastases in a single organ or in multiple organs |

TABLE 24
Small Cell Lung Cancer Anatomic stage/prognostic groups

| | | | |
|---|---|---|---|
| Stage | T | N | M |

Limited disease

| | | | |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| IIIA | T2a | N2 | M0 |
| | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| IIIC | T3 | N3 | M0 |

Extensive disease

| | | | |
|---|---|---|---|
| IVA | T Any | N Any | M1a |
| | T Any | N Any | M1b |
| IVB | T Any | N Any | M1c |

TABLE 25
Breast Cancer Primary tumor (T)

| | |
|---|---|
| TX | Primary tumor cannot be assessed |
| T0 | No evidence of primary tumor |
| Tis | Carcinoma in situ |
| Tis (DCIS) | Ductal carcinoma in situ |
| Tis | Paget disease of the nipple NOT associated with invasive carcinoma and/or carcinoma in |
| (Paget) | situ (DCIS) in the underlying breast parenchyma. Carcinomas in the breast parenchyma |
| | associated with Paget disease are categorized on the basis of the size and characteristics of |
| | the parenchymal disease, although the presence of Paget disease should still be noted |
| T1 | Tumor ≤20 mm in greatest dimension |
| T1mi | Tumor ≤1 mm in greatest dimension |
| T1a | Tumor >1 mm but ≤5 mm in greatest dimension (round any measurement >1.0-1.9 mm to |
| | 2 mm) |
| T1b | Tumor >5 mm but ≤10 mm in greatest dimension |
| T1c | Tumor >10 mm but ≤20 mm in greatest dimension |
| T2 | Tumor >20 mm but ≤50 mm in greatest dimension |
| T3 | Tumor >50 mm in greatest dimension |
| T4 | Tumor of any size with direct extension to the chest wall and/or to the skin (ulceration or |
| | skin nodules), not including invasion of dermis alone |
| T4a | Extension to chest wall, not including only pectoralis muscle adherence/invasion |
| T4b | Ulceration and/or ipsilateral satellite nodules and/or edema (including peaud'orange) of |
| | the skin, which do not meet the criteria for inflammatory carcinoma |
| T4c | Both T4a and T4b |
| T4d | Inflammatory carcinoma |

Breast Cancer Regional lymph nodes (N)
Clinical

| | |
|---|---|
| cNX | Regional lymph nodes cannot be assessed (e.g., previously removed) |
| cN0 | No regional lymph node metastasis (on imaging or clinical examination) |
| cN1 | Metastasis to movable ipsilateral level I, II axillary lymph node(s) |
| cN1mi | Micrometastases (approximately 200 cells, larger than 0.2 mm, but none larger than 2.0 |
| | mm) |
| cN2 | Metastases in ipsilateral level I, II axillary lymph nodes that are clinically fixed or matted; |
| | or in ipsilateral internal mammary nodes in the absence of clinically evident axillary |
| | lymph node metastases |
| cN2a | Metastases in ipsilateral level I, II axillary lymph nodes fixed to one another (matted) or to |
| | other structures |
| cN2b | Metastases only in ipsilateral internal mammary nodes and in the absence of axillary |
| | lymph node metastases |
| cN3 | Metastases in ipsilateral infraclavicular (level III axillary) lymph node(s), with or without |
| | level I, II axillary node involvement, or in ipsilateral internal mammary lymph node(s) |
| | with level I, II axillary lymph node metastasis; or metastases in ipsilateral supraclavicular |
| | lymph node(s), with or without axillary or internal mammary lymph node involvement |
| cN3a | Metastasis in ipsilateral infraclavicular lymph node(s) |
| cN3b | Metastasis in ipsilateral internal mammary lymph node(s) and axillary lymph node(s) |
| cN3c | Metastasis in ipsilateral supraclavicular lymph node(s) |

Breast Cancer Pathologic (pN)

| | |
|---|---|
| pNX | Regional lymph nodes cannot be assessed (for example, previously removed, or not |
| | removed for pathologic study) |
| pN0 | No regional lymph node metastasis identified histologically, or isolated tumor cell clusters |
| | (ITCs) only. Note: ITCs are defined as small clusters of cells ≤0.2 mm, or single tumor |
| | cells, or a cluster of <200 cells in a single histologic cross-section; ITCs may be detected |
| | by routine histology or by immunohistochemical (IHC) methods; nodes containing only |
| | ITCs are excluded from the total positive node count for purposes of N classification but |
| | should be included in the total number of nodes evaluated |
| pN0(i) | No regional lymph node metastases histologically, negative IHC |
| pN0(i+) | ITCs only in regional lymph node(s) |
| pN0(mol−) | No regional lymph node metastases histologically, negative molecular findings (reverse |
| | transcriptase polymerase chain reaction [RT-PCR]) |
| pN0(mol+) | Positive molecular findings by RT-PCR; no ITCs detected |
| pN1 | Micrometastases; or metastases in 1-3 axillary lymph nodes and/or in internal mammary |
| | nodes; and/or in clinically negative internal mammary nodes with micrometastases or |
| | macrometastases by sentinel lymph node biopsy |
| pN1mi | Micrometastases (200 cells, >0.2 mm but none >2.0 mm) |
| pN1a | Metastases in 1-3 axillary lymph nodes (at least 1 metastasis >2.0 mm) |
| pN1b | Metastases in ipsilateral internal mammary lymph nodes, excluding ITCs, detected by |
| | sentinel lymph node biopsy |
| pN1c | Metastases in 1-3 axillary lymph nodes and in internal mammary sentinel nodes (i.e., |
| | pN1a and pN1b combined) |
| pN2 | Metastases in 4-9 axillary lymph nodes; or positive ipsilateral internal mammary lymph |
| | nodes by imaging in the absence of axillary lymph node metastases |
| pN2a | Metastases in 4-9 axillary lymph nodes (at least 1 tumor deposit >2.0 mm) |
| pN2b | Clinically detected*¹ metastases in internal mammary lymph nodes with or without |
| | microscopic confirmation; with pathologically negative axillary lymph nodes |
| pN3 | Metastases in ≥10 axillary lymph nodes; or in infraclavicular (level III axillary) lymph |
| | nodes; or positive ipsilateral internal mammary lymph nodes by imaging in the presence of |
| | one or more positive level I, II axillary lymph nodes; or in >3 axillary lymph nodes and |
| | micrometastases or macrometastases by sentinel lymph node biopsy in clinically negative |
| | ipsilateral internal mammary lymph nodes; or in ipsilateral supraclavicular lymph nodes |
| pN3a | Metastases in ≥10 axillary lymph nodes (at least 1 tumor deposit >2.0 mm); or metastases |
| | to the infraclavicular (level III axillary lymph) nodes |
| pN3b | pN1a or pN2a in the presence of cN2b (positive internal mammary nodes by imaging) or |
| | pN2a in the presence of pN1b |
| pN3c | Metastases in ipsilateral supraclavicular lymph nodes |

Breast Cancer Distant metastasis (M)

| | |
|---|---|
| M0 | No clinical or radiographic evidence of distant metastasis |
| cM0(i+) | No clinical or radiographic evidence of distant metastases in the presence of tumor cells or |
| | deposits no larger than 0.2 mm detected microscopically or by molecular techniques in |
| | circulating blood, bone marrow, or other nonregional nodal tissue in a patient without |
| | symptoms or signs of metastasis |
| cM1 | Distant metastases detected by clinical and radiographic approaches |
| pM1 | Any histologically proven metastases in distant organs; or if in non-regional nodes, |
| | metastases >0.2 mm |

TABLE 26
Breast Cancer Histologic grade (G)

| | |
|---|---|
| GX | Grade cannot be assessed |
| G1 | Low combined histologic grade (favorable) |
| G2 | Intermediate combined histologic grade (moderately favorable) |
| G3 | High combined histologic grade (unfavorable) |

TABLE 27
Breast Cancer Anatomic stage/prognostic groups

| Stage | T | N | M |
|---|---|---|---|
| 0 | Tis | N0 | M0 |
| IA | T1 | N0 | M0 |
| IB | T0 | N1mi | M0 |
| | T1 | N1mi | M0 |
| IIA | T0 | N1 | M0 |
| | T1 | N1 | M0 |
| | T2 | N0 | M0 |
| IIB | T2 | N1 | M0 |
| | T3 | N0 | M0 |
| IIIA | T0 | N2 | M0 |
| | T1 | N2 | M0 |
| | T2 | N2 | M0 |
| | T3 | N1 | M0 |
| | T3 | N2 | M0 |
| IIIB | T4 | N0 | M0 |
| | T4 | N1 | M0 |
| | T4 | N2 | M0 |
| IIIC | Any T | N3 | M0 |
| IV | Any T | Any N | M1 |

Methods provided herein may allow for early detection cancer or for detection of non-metastatic cancer. Examples of cancers that may be detected in accordance with a method disclosed herein include, without limitation, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Early Cancer Detection Using Multi-Omics Approach Including Epigenetic Signal from Ultra-Small Fragments

Background

Genetic and epigenetic signals from plasma cell-free DNA (cfDNA) as well as proteins have been shown to detect cancer early. Circular Ligation and Amplification (CLAmp-seq) has been shown to detect mutations very sensitively and demonstrated higher performance than molecular barcode-based methods. The same single strand based technology yielded a much larger proportion of small fragments (<100 bp) than traditional methods typically relying on double stranded ligation, allowing us to investigate epigenetic signature of cancer in ultra-short fragments. The data below measures the performance using a multi-omics approach of these genetic and epigenetic changes as well as proteins in detecting colorectal cancer (CRC), ovarian cancer (OC) and hepatocellular carcinoma (HCC).

Methods

A healthy and a late stage cancer sample were assessed by whole genome sequencing (WGS) using CLAmp-seq and traditional double stranded library preparation. Then cfDNA was analyzed from plasma samples of 731 patients, including 69 CRC, 57 HCC, 49° C. patients and 556 age matched healthy individuals. Out of the diseased samples, the numbers for stages I-IV are 49, 39, 71, and 16, respectively. CLAmp-seq WGS was performed on 58 healthy and 66 cancer samples to discover cancer epigenetic signature. In addition, all the samples were analyzed for a panel of proteins and a CLAmp-seq targeted panel that includes known mutation sites.

Results

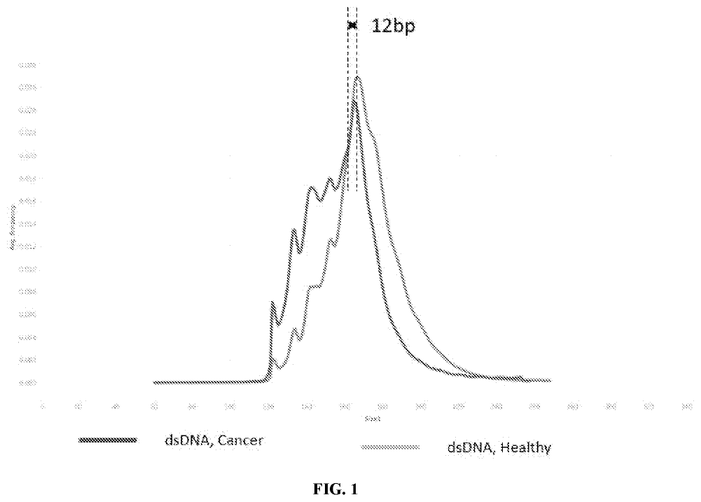

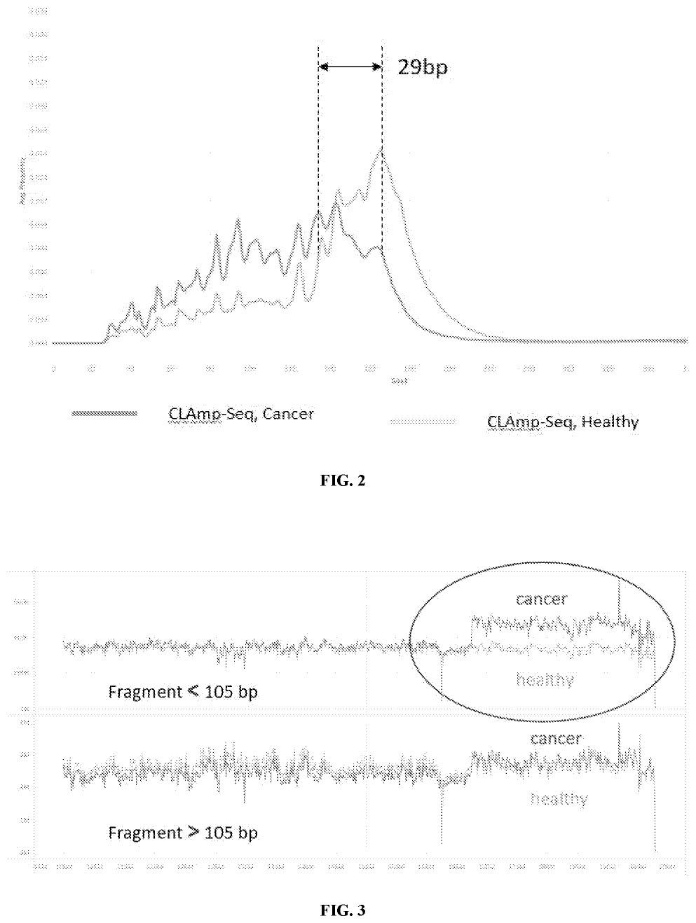

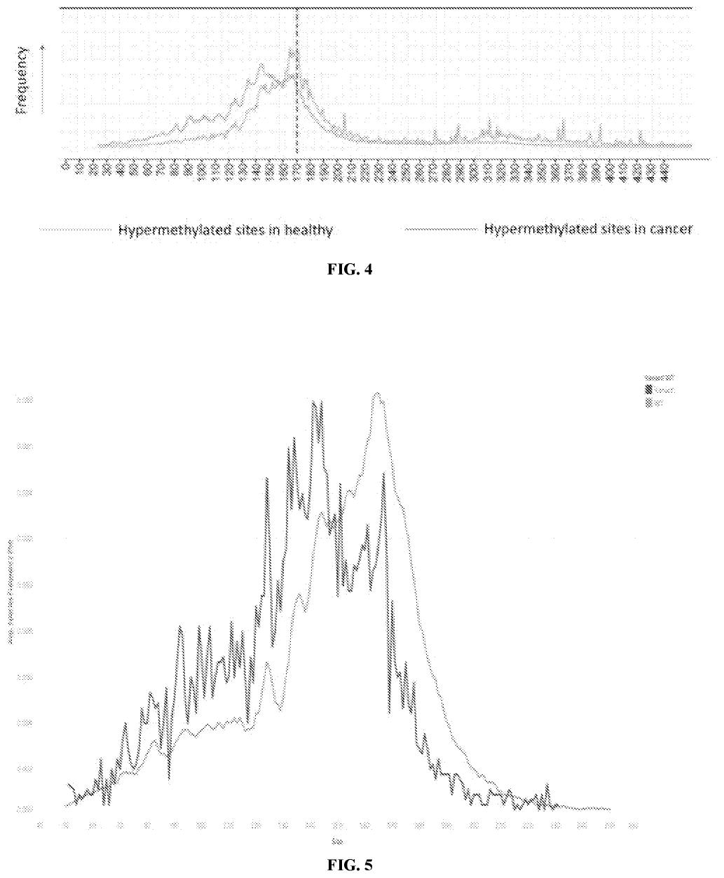

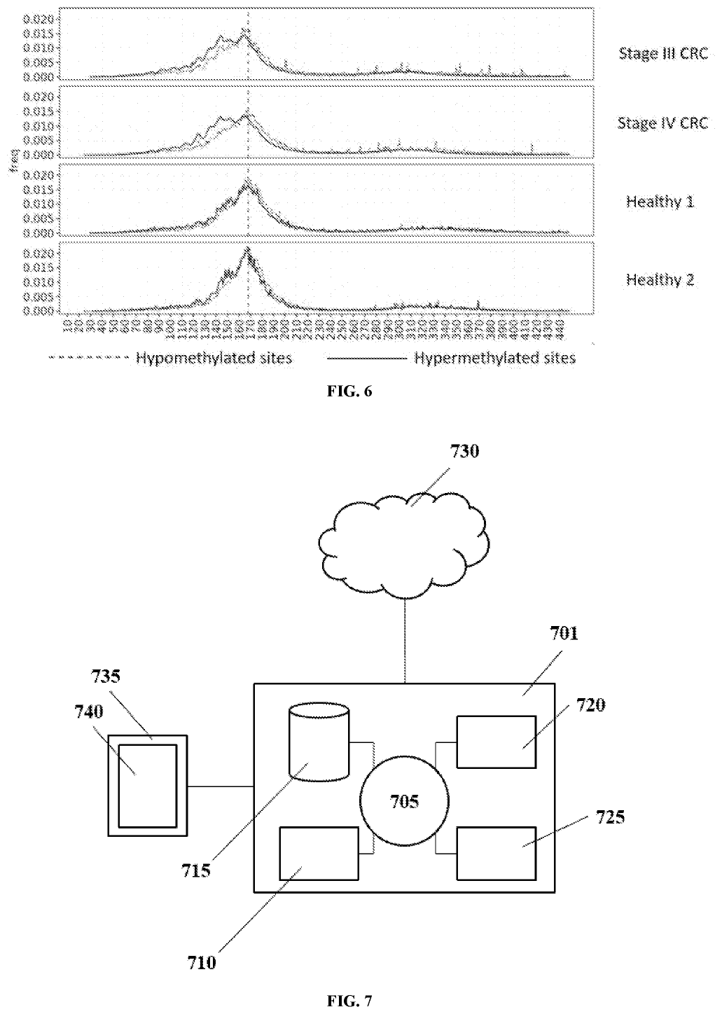

Using CLAmp-Seq in late stage cancer showed 33% of its fragments as smaller than 100 bp compared to 15% in healthy and <1% in late stage by double stranded library prep. In addition, the difference in fragment size between late stage cancer and healthy was 29 bp using CLAmp-Seq and 12 bp using traditional double stranded prep (FIG. 1, FIG. 2). Next, epigenetic signature specific to cancer was detected on the small fragments using CLAmp-Seq. Using data from whole genome analysis it was demonstrated that a performance using the epigenetic signature alone of 50% sensitivity at 97% specificity (FIG. 4). Combined with mutations and proteins we obtained at specificity of 97% sensitivities of 50%, 88%, 88%, and 100% in stage I, II, III, and IV, respectively. At the same 97% specificity we obtained the sensitivities of 73%, 100%, and 85% in CRC, OC, and HCC, respectively.

Conclusions

It has been demonstrated herein that CLAmp-Seq detects small fragments that are enriched in cancer. Predictive epigenetic signature was found in these small fragments. When combined with mutations and proteins a performance of 80% sensitivity at 97% specificity was obtained.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for nucleic acid analysis, comprising: (a) preparing a first single-stranded deoxynucleic acid (DNA) library from a first plurality of nucleic acid molecules, said first plurality of nucleic acid molecules comprising single-stranded DNA and double-stranded DNA derived from a subject; (b) preparing a second single-stranded DNA library from a second plurality of nucleic acid molecules, said second plurality of nucleic acid molecules comprising single-stranded DNA and double-stranded DNA derived from a control; (c) using said first single-stranded DNA library to measure a first size distribution for at least a sub set of said first plurality of nucleic acid molecules; (d) using said second single-stranded DNA library to measure a second size distribution for at least a subset of said second plurality of nucleic acid molecules; (e) using said first size distribution and said second size distribution to determine a difference in size between said first size distribution and said second size distribution.

2. The method of claim 1, wherein (a) comprises denaturing said first plurality of nucleic acid molecules and/or (b) comprises denaturing said second plurality of nucleic acid molecules.

3. (canceled)

4. The method of claim 1, wherein (a) comprises ligating an adapter to 5′ ends, 3′ ends or 5′ and 3′ ends of individual single stranded DNA molecules of said first plurality of nucleic acid molecules using a DNA ligase specific for single stranded DNA and/or (b) comprises ligating an adapter to 5′ ends, 3′ ends or 5′ and 3′ ends of individual single stranded DNA molecules of said second plurality of nucleic acid molecules using a DNA ligase specific for single stranded DNA.

5. (canceled)

6. The method of claim 1, wherein (a) comprises (i) circularizing individual single stranded DNA molecules of said first plurality of nucleic acid molecules to form a first plurality of circular nucleic acid molecules; and (ii) amplifying said first plurality of circular nucleic acid molecules to yield a first plurality of amplified nucleic acid molecules and/or (b) comprises (i) circularizing individual single stranded DNA molecules of said second plurality of nucleic acid molecules to form a second plurality of circular nucleic acid molecules; and (ii) amplifying said second plurality of circular nucleic acid molecules to yield a second plurality of amplified nucleic acid molecules.

7.-12. (canceled)

13. The method of claim 1, further comprising enriching said first plurality of nucleic acid molecules or said second plurality of nucleic acid molecules for one or more target sequences.

14. The method of claim 13, wherein said enriching is performed with one or more primers or capture probes.

15. The method of claim 13, wherein said enriching is performed with one or more antibodies or fragments thereof.

16. The method of claim 1, wherein measuring said size distribution for said first single-stranded DNA library or said second single-stranded DNA library comprises sequencing said first single-stranded DNA library or said second single-stranded DNA library.

17. (canceled)

18. The method of claim 1, further comprising identifying an individual nucleic acid molecule of said single-stranded DNA library or said second single stranded library as having a genomic feature.

19. The method of claim 18, wherein said genomic feature comprises an epigenetic modification, selected from the group consisting of methylation, phosphorylation, ubiquitination, sumoylation, acetylation, rib osylation, citrullination, and fragmentation.

20. (canceled)

21. The method of claim 18, said genomic feature comprises a copy number variation (CNV), a single nucleotide variant (SNV), an insertion, a deletion, a translocation, or a combination thereof.

22.-24. (canceled)

25. The method of claim 1, wherein said first plurality of nucleic acids comprises tumor nucleic acids and/or wherein said second plurality of nucleic acids is derived from a healthy control.

26. (canceled)

27. The method of claim 1, wherein said subject is determined to be at risk of or to have a disease when said difference is greater than a predetermined threshold and/or wherein said subject is determined to be at risk of or to have a disease when an average of said first size distribution is less than an average of said second size distribution.

28. (canceled)

29. The method of claim 27, wherein said disease is cancer.

30. (canceled)

31. The method of claim 27, further comprising administering a therapeutic to said subject, recommending additional cancer monitoring to said subject, or using said difference to monitor said subject for a progression or a regression of said disease.

32.-33. (canceled)

34. A method for nucleic acid analysis, comprising: (a) preparing a single-stranded deoxynucleic acid (DNA) library from a plurality of nucleic acid molecules said plurality comprising single-stranded DNA and double-stranded DNA derived from a subject; (b) identifying an individual nucleic acid molecule of said single-stranded DNA library as having a genomic feature; (c) measuring a size for said individual nucleic acid molecule; (d) associating said genomic feature with a disease based on said size of said individual nucleic acid molecule.

35. The method of claim 34, wherein (a) comprises denaturing said plurality of nucleic acid molecules and/or wherein (a) comprises ligating an adapter to 5′ ends, 3′ ends or 5′ and 3′ ends of individual single stranded DNA molecules of said plurality of nucleic acid molecules using a DNA ligase specific for single stranded DNA.

36. (canceled)

37. The method of any one of claims 34 to 36, wherein (a) comprises (i) circularizing individual single stranded DNA molecules of said plurality of nucleic acid molecules to form a plurality of circular nucleic acid molecules; and (ii) amplifying said plurality of circular nucleic acid molecules to yield a plurality of amplified nucleic acid molecules.

38.-49. (canceled)

50. The method of claim 34, wherein said genomic feature comprises an epigenetic modification selected from the group consisting of methylation, phosphorylation, ubiquitination, sumoylation, acetylation, ribosylation, citrullination, and fragmentation.

51. (canceled)

52. The method of claim 34, wherein said genomic feature comprises a copy number variation (CNV), a single nucleotide variant (SNV), an insertion, a deletion, a translocation, or a combination thereof.

53.-64. (canceled)

65. A method for nucleic acid analysis, comprising: (a) preparing a single-stranded deoxynucleic acid (DNA) library from a plurality of nucleic acid molecules said plurality comprising single-stranded DNA and double-stranded DNA derived from a subject; (b) identifying an individual nucleic acid molecule of said single-stranded DNA library as having a genomic feature; (c) identifying at least a 5′ end or a 3′ end for said individual nucleic acid molecule; (d) associating said genomic feature with a disease based on said 5′ end or said 3′ end of said individual nucleic acid molecule.

66.-171. (canceled)
